(19) United States
(12) Patent Application Publication
(10) Pub. No.: US 2022/0409527 A1
(43) Pub. Date: Dec. 29, 2022

(54) TREATMENT OF PERIODONTITIS VIA INDUCTION OF M2 MACROPHAGES

(71) Applicant: UNIVERSITY OF PITTSBURGH - OF THE COMMONWEALTH SYSTEM OF HIGHER EDUCATION, PITTSBURGH, PA (US)

(72) Inventor: Charles S. SFEIR, Wexford, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 17/787,672

(22) PCT Filed: Jan. 21, 2021
(86) PCT No.: PCT/US2021/014285

§ 371 Date: Jun. 21, 2022

Related U.S. Application Data
(60) Provisional application No. 62/963,632, filed on Jan. 21, 2020.

(51) Int. Cl. A61K 9/00; C07K 16/24; A61K 38/19; A61P 1/02; A61K 9/16

(52) U.S. Cl. CPC A61K 9/0063; C07K 16/247; A61K 38/195; C07K 16/244; A61P 1/02; A61K 9/1647

(57) ABSTRACT

The present invention relates to systems and methods for the treatment of conditions of chronic inflammation, such as periodontitis, that includes the use of sustained release microparticles to locally deliver a compound or agent to reduce inflammation of tissue, such as periodontal tissue, and/or inflammatory mediators. The microparticles are loaded with a compound or agent selected from the group consisting of C-C motif chemokine ligand 2 (CCL2), interleukin 4 (IL-4), anti-interleukin 17 (IL17 antibody), and mixtures or blends thereof

### CROSS-REFERENCE TO RELATED APPLICATIONS

This application claims priority under 35 U.S.C. § 119(e) from U.S. provisional patent application No. 62/963,632, entitled “TREATMENT OF PERIODONTITIS VIA INDUCTION OF M2 MACROPHAGES” and filed on Jan. 21, 2020, the contents of which are incorporated herein by reference.

### GOVERNMENT CONTRACT

This invention was made with government support under grants DE025735 and DE026915 awarded by the National Institutes of Health (NIH). The government has certain rights in the invention.

### BACKGROUND OF THE INVENTION

1. Field of the Invention

The present invention relates to compositions and methods for the treatment of conditions of chronic inflammation that includes the use of sustained release microparticles to locally deliver a compound to reduce inflammation of tissue and/or inflammatory mediators.

### 2. Description of the Related Art

It is known that chronic inflammation of tissue in a body of a human subject causes progressive destruction of the tissue. For example, periodontal disease (PD) and rheumatoid arthritis are both characterized by the progressive destruction of tissue.

Periodontal disease (PD) is a chronic destructive inflammatory disease of the tooth-supporting structures forming the periodontium that leads to tooth loss at its advanced stages. Although the disease is initiated by a complex organization of oral microorganisms in the form of a plaque biofilm, it is the uncontrolled immune response to periodontal pathogens that fuels periodontal tissue destruction. Recently, Interleukin (IL)-17 has been identified as a key cytokine in the pathogenesis of PD. Despite its well documented role in host defense against invading pathogens at oral barrier sites, IL-17 mediated signaling can also lead to a detrimental inflammatory response that causes periodontal tissue destruction and bone resorption.

Although PD is triggered by keystone pathogens and pathobionts that colonize the tooth surface, it is the dysregulated host immune response against those pathogens that cause periodontal tissue destruction. This dysregulated host response is fueled by an array of mediators that induce inflammatory osteolysis in the periodontium. Among those mediators are well-established pro-inflammatory cytokines, such as Interleukin-6 (IL-6) and tumor necrosis factor-alpha (TNF-a). Both cytokines and others are released by immune (e.g. macrophages) and structural (e.g. fibroblasts) cells in response to bacterial invasion in the periodontium, and have been associated with enhanced osteoclastic activity and progressive alveolar bone loss in animal models of PD.

More recently, Interleukin-17 (IL-17A) has been implicated as a key driver of pathogenic host response in a number of chronic inflammatory conditions where bone loss is a characteristic feature, such as psoriatic arthritis and PD. In both conditions, chronic expansion and activation of Th17 cells, the major source of IL-17A, under the influence of locally upregulated IL-23 and IL-6 results in progressive bone destruction. A number of mechanistic studies have indicated that IL-17A mediates bone loss by influencing osteoblasts, osteocytes and osteoclasts functions. In osteoblasts cultures, IL-17A can stimulate the expression of the major osteoclast-differentiating factor RANKL in a dose dependent manner. Similarly, a recent report indicated that the inducing effect of IL-17A on RANKL extends to osteocytes using an animal model of hyperparathyroidism-induced bone loss. In osteoclasts, IL-17A can either promote the generation of osteoclasts from monocytes in the absence of RANKL, or enhance the sensitivity of osteoclasts precursors to RANKL by upregulating the expression of its receptor. In addition to the interaction of IL-17A with cells that are directly involved in the bone remodeling process, IL-17A can indirectly influence bone loss by acting as a potent inducer of other pro-osteoclastogenic cytokines, such as IL-6, TNF-α and IL-1β. Thus, it appears that the overall effect of IL-17A upregulation in chronic inflammatory conditions of bone is destructive, where it supports uncoupled bone remodeling by favoring osteoclastic activity through direct and indirect mechanisms.

Interestingly, numerous studies have pointed out a protective role for IL-17-mediated inflammation against pathogen induced tissue damage. In the oral environment, IL-17A signaling has been deemed crucial for host defense against P. Gingivalis induced bone loss, and Candida albicans infection in mice. Both models of oral infection are considered acute forms of disease. In this specific situation, IL-17A mediated protection can be explained by its vital role in orchestrating neutrophils expansion and recruitment to the infection site.

The aforementioned reports indicate that the end result of IL-17 driven inflammation depends on the nature of the inflammatory response produced in tissue as determined by the animal model employed. In acute infection models (acute oropharyngeal candidiasis and P. Gingivalis induced PD), the protective role of IL-17A is illustrated by its neutrophil-recruiting ability in an attempt to limit pathogen invasion. Conversely, the destructive effects of IL-17A predominate in chronic models of inflammatory bone loss (collagen-induced arthritis and ligature-induced PD), owing to its direct and indirect pro-osteoclastogenic effects.

Considering the destructive role of IL-17A in chronic inflammatory diseases, anti-IL-17 therapy has been investigated with varying degrees of success. In this respect, clinical trials of anti-IL17A therapy showed promise for alleviating psoriatic arthritis. However, clinical efficacy of IL-17A blockade was questionable for rheumatoid arthritis.

Known standards of care for periodontitis have focused on reducing microbial load in the periodontium by periodic debridement of dental plaque and local delivery of antibiotics. However, this approach does not effectively address the uncontrolled host immune response that is responsible for most of the damage and disease progression.

There is a need in the art to develop treatment systems and methods to effectively halt progression of periodontitis and related inflammatory tissue conditions, by inhibiting inflammatory bone loss and promoting inflammation resolution. In accordance with the invention, there is a need to develop sustained release systems and methods capable of reducing the effective amount of inflammatory activity, e.g., resulting from free IL-17 in tissues, which include IL-17 antibody (Ab) incorporated in poly(lactic-co-glycolic) acid microparticles (MP).

Additionally, there is a need in the art to develop sustained release systems, e.g., poly(lactic-co-glycolic) acid microparticles (MP), to locally deliver, e.g., by injection, interleukin-4 (IL-4) and/or C-C motif chemokine ligand 2 (CCL2) and/or anti-interleukin-17A (IL-17A) antibody into inflamed tissues. IL-4 is a well- established T-helper-2 cytokine that induces the polarization of macrophages towards the M2 phenotype by acting through the IL-4 receptor alpha, whereas CCL2 is known to promote chemotaxis of MO or M1, or M2 phenotype macrophages to the inflamed site and induce their polarization to the anti-inflammatory pro-resolving M2 phenotype. It is contemplated that this approach is effective in halting the progression of tissue destruction by inhibiting inflammatory bone loss and promoting inflammation resolution.

It is further contemplated that treatment compositions and methods of the invention are effective to control or mitigate various conditions associated with inflammatory bone loss, such as, but not limited to, PD and rheumatoid arthritis.

### SUMMARY OF THE INVENTION

In one aspect, the invention provides a microparticle that includes a degradable polymer and a compound selected from the group consisting of C-C motif chemokine ligand 2 (CCL2), interleukin 4 (IL-4), anti-interleukin 17 (IL17 antibody), and mixtures or blends thereof, wherein the degradable polymer is loaded with the compound, and the microparticle is structured to provide a sustained, controlled release of said compound from said degradable polymer.

The degradable polymer may include poly(lactic-co-glycolic) acid.

The microparticle can be administered in a therapeutically effective amount to a patient.

The microparticle may be an active ingredient in a composition. In certain embodiments, the microparticle is administered by locally injecting the composition in the periodontium of a patient.

The microparticle may be effective to reduce or eliminate periodontal disease and reduce inflammation. When the compound is anti-interleukin 17 (IL-17 antibody), the microparticle may be effective to prevent osteoclast formation for the prevention of bone loss. When the compound is at least one of C-C motif chemokine ligand 2 (CCL2) and interleukin 4 (IL-4), the microparticle may be effective to recruit M2 macrophages and convert M1 macrophages to M2 for acute prevention of inflammation.

In another aspect, the invention provides a pharmaceutical composition that includes loaded microparticles, which include degradable polymer and a compound selected from the group consisting of C-C motif chemokine ligand 2 (CCL2), interleukin 4 (IL-4), anti-interleukin 17 (IL-17 antibody), and mixtures or blends thereof; and at least one ingredient selected from the group consisting of a pharmaceutically acceptable carrier, an adjuvant and an excipient.

In still another aspect, the invention provides a method of administering a therapeutically effective amount of a compound selected from the group consisting of C-C motif chemokine ligand 2 (CCL2), interleukin 4 (IL-4), anti-interleukin 17 (IL-17 antibody), and mixtures or blends thereof, to a subject in need of reduced tissue inflammation, wherein the compound is locally delivered at the site of the tissue inflammation in a sustained release form, and the sustained release form comprises microparticles loaded with the compound.

In certain embodiments, the tissue inflammation is periodontal disease.

In yet another aspect, the invention provides a method of treating a patient with tissue inflammation. The method includes forming microparticles including degradable polymer and a compound selected from the group consisting of C-C motif chemokine ligand 2 (CCL2), interleukin 4 (IL-4), anti-interleukin 17 (IL-17 antibody), and mixtures or blends thereof, wherein the degradable polymer is loaded with the compound; and administering a therapeutically effective amount of the microparticles locally to the tissue inflammation, wherein the microparticles are structured to provide a sustained, controlled release of said compound from said degradable polymer to the tissue inflammation.

The aforementioned method can be effective in clinical scenarios, comprising:

preventative, wherein tissue is healthy, and disease is induced but microparticles are also delivered, the disease is prevented from occurring;

interventional, wherein tissue is inflamed, disease is in progress, then the microparticles are injected, and the disease is halted or stopped; and

wherein the cause of the disease is removed, removal of the ligature, then microparticles are injected, and healing and regeneration at the site is enhanced.

### BRIEF DESCRIPTION OF THE DRAWINGS

FIGS. 1A, 1B and 1C are characterizations of PLGA microparticles encapsulating IL-17A antibody in accordance with certain embodiments of the invention; cumulative fraction of IL-17Ab released from PLGA MPs for 14 days, determined by ELISA (FIG. 1A); Scanning Eletron Microscopy image of IL-17AbMP, ×2000 (FIG. 1B) and ×5500 (FIG. 1C).

FIG. 2 is an expression of IL-17A gene in the gingiva around the ligated maxillary second molar during the course of 8 days ligature induced periodontitis model, in accordance with certain embodiments of the invention. Data represents the mean fold change analyzed by delta-delta CT method from 5 mice followed by Holm's Sidak's multiple comparisons test (*p<0.05 by one-way ANOVA followed by Tukey's multiple-comparisons test).

FIGS. 3A, 3B and 3C are microtomographic (microCT) evaluations of alveolar bone loss in mice, in accordance with certain embodiments of the invention. Microparticles (MPs) were injected into gingival tissue surrounding the ligated tooth on either day 0 or day 2, after ligature placement. Representative 2D microCT images from sagittal and transaxial slices of mice hemi-maxilla: healthy group, untreated group, and groups treated with IL-17AbMP day 0 and day 2. Quantification of alveolar bone loss represented by the linear bone loss between the CEJ and ABC (dashed red lines) along the interdental (FIG. 3A), buccal (FIG. 3B) and palatal (FIG. 3C) sides. Values (mean±SD) obtained from 5-6 animals per group; *p<0.05 by one-way ANOVA followed by Tukey's multiple-comparisons test.

FIGS. 4A, 4B, 4C, 4D, 4E and 4F show the effect of Anti-IL-17A MPs on the number of alveolar bone-associated osteoclasts, in accordance with certain embodiments of the invention. (FIGS. 4A, 4B, 4C and 4D) Histological representation of healthy, untreated, and IL-17AMPs—day 0 and day 2 groups. Hemi-maxilla samples were stained for TRAP, as described in Material and Methods section. The arrows show TRAP-positive multinucleated osteoclasts associated to alveolar bone (Magnification: 400×. Scale bar: 100 μm). (FIGS. 4E and 4F) Quantification of TRAP-positive multinucleated alveolar bone-associated osteoclasts on the mesial, distal and furcation areas. Anti-IL-17A MPs at day 2 significantly decreased the number of osteoclasts per mm² in the alveolar bone in comparison to untreated group. Values (mean±SD) obtained from 6 animals per group; *p<0.05 by one-way ANOVA followed by Tukey's multiple-comparisons test.

FIGS. 5A, 5B, 5C, 5D, 5E and 5F is an expression of pro-inflammatory markers in periodontal tissues of mice, in accordance with certain embodiments of the invention. IL-6, TNF-α and RANKL expression in the periodontal tissues was compared by the value of 2^{(-ΔΔCt)} (n=5 mice). Microparticles were injected into the maxillary gingiva on day 2 (buccal and palatal sides) after ligation and the biochemical markers were assessed at 4^{th} day (FIGS. 5A, 5B and 5C) and 8^{th} day (FIGS. 5D, 5E and 5F) after ligature placement. Periodontal tissues from untreated group showed an increase in IL-6 and RANKL levels at 4 days. Anti-IL-17A MPs injection decreased the levels of IL-6 when evaluated in the 4^{th} but not in the 8^{th} day after ligature placement. RANKL expression did not change by IL-17A MPs delivery at 4 or 8 days. Ligature placement and 17A MPs injection did not affect TNF-α expression in the periodontal tissues at both timepoints. (*p<0.05 by one-way ANOVA followed by Tukey's multiple-comparisons test).

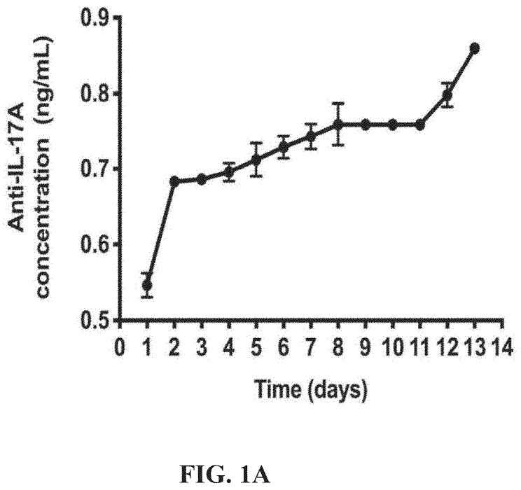

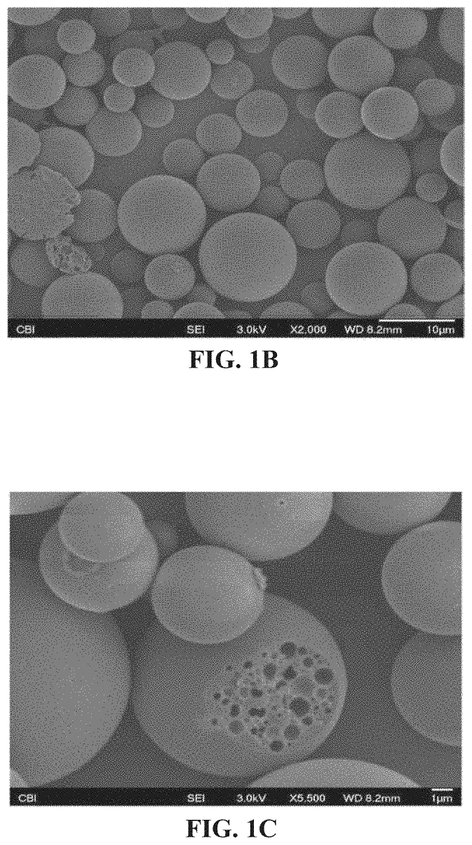

Supplemental FIGS. 1A and 1B show the effect of blank PLGA MPs injection in total alveolar bone loss and osteoclast counting, respectively, in accordance with certain embodiments of the invention. Total bone is the sum of measurements between the CEJ and ABC on the buccal, palatal and interdental sides. Total osteoclasts represents the sum of TRAP-positive cells on the mesial, distal and furcation areas. Total bone loss and osteoclasts count were significantly higher in the untreated and blank MPs groups. Both the untreated and blank MPs groups exhibited similar trends of bone loss and osteoclastic activity with no statistically significant difference. Values (mean±SD) from 6 animals per group; *P<0.001 by one-way ANOVA followed by Tukey's multiple-comparisons test.

FIGS. 6A, 6B, 6C and 6D include a SEM image that shows uniformly spherical MPs with moderate surface, a plot of ELISA release profile data that shows the IL-4 MPs continued sustainable release, and in-vitro functional assay data that show conditioned medium containing IL-4 released from MPs upregulated arginase activity (M2 marker) in RAW cells and increased expression of CD206 (surface marker of M2 macrophages) in the same manner as soluble IL-4, respectively, in accordance with certain embodiments of the invention.

FIGS. 7A and 7B are microCT analysis data that show both CCL2 and IL-4 MPs inhibited bone loss significantly in both the preventative and interventional models, respectively, in accordance with certain embodiments of the invention.

FIG. 8 is microCT analysis data that shows IL-4 MP accelerated bone gain in the reparative model, in accordance with certain embodiments of the invention.

FIG. 9 includes plots that show Q-PCR analysis of in vivo expression of M2-like macrophages, M1-like macrophages, bone resorption and regulatory T-cell markers in gingival tissues of mice where ligature periodontitis was induced and IL-4 or CCL2 MPs were delivered, in accordance with certain embodiments of the invention.

FIGS. 10A and 10B are plots that show FACS analysis of gingival macrophages following 7 days ligature induced PD; ligature PD increases the number of M1-like macrophages (CD45+CD11b+CD86+) in the gingiva around the ligated tooth (A) and decreases the number of gingival M2-like macrophages (CD45+CD11b+CD206+) (B), in accordance with certain embodiments of the invention.

FIGS. 11A and 11B are plots that show QPCR analysis of macrophages polarization, inflammatory and osteoclastic differentiation makers at 4 (FIG. 11A) and 8 days (FIG. 11B) post ligature placement with or without PLGA MPs local delivery in the gingiva surrounding ligated maxillary molars; one-way ANOVA followed by Tukey's post hoc test (n=5 mice), was used; statistical significance was considered at P<0.05, in accordance with certain embodiments of the invention.

FIGS. 12A, 12B and 12C are micro-computed tomography analysis of alveolar bone loss around ligated second molar following preventive M2 induction therapy; FIG. 12A is a timeline of the preventive therapeutic approach experiment; FIG. 12B is a quantification of the distance between the alveolar bone crest (ABC) to cemento-enamel junction (CEJ) on the proximal, buccal and palatal aspects, as well as the sum of all aspects; one-way ANOVA followed by Tukey's post hoc test (n=6 mice), was used; statistical significance was considered at P<0.05; and FIG. 12C shows representative 2D images of the sagittal view of maxillary molar teeth.

FIGS. 13A, 13B and 13C are micro-computed tomography analysis of alveolar bone loss around ligated second molar following preventive M2 induction therapy. 13A is a timeline of the interventional therapeutic approach experiment; 13B is a quantification of the distance between the alveolar bone crest (ABC) to cemento-enamel junction (CEJ) on the proximal, buccal and palatal aspects, as well as the sum of all aspects; one-way ANOVA followed by Tukey's post hoc test (n=6 mice), was used; statistical significance was considered at P<0.05; FIG. 13C shows representative 2D images of the sagittal view of maxillary molar teeth.

FIGS. 14A, 14B and 14C are micro-computed tomography analysis of alveolar bone gain around second molar following ligature removal and reparative M2 induction therapy; FIG. 14A is a timeline of the reparative therapeutic approach experiment; FIG. 14B is a quantification of distance between the alveolar bone crest (ABC) to cemento-enamel junction (CEJ) on the proximal, buccal and palatal aspects, as well as the sum of all aspects; one-way ANOVA followed by Tukey's post hoc test (n=6 mice), was used; statistical significance was considered at P<0.05; FIG. 14C shows representative 2D images of the sagittal view of maxillary molar teeth.

### DETAILED DESCRIPTION OF EXEMPLARY EMBODIMENTS

As used herein, the singular form of “a”, “an”, and “the” include plural references unless the context clearly dictates otherwise.

“Inhibiting” refers to inhibiting the full development of a disease or condition. “Inhibiting” also refers to any quantitative or qualitative reduction in biological activity or binding, relative to a control.

“Microparticle” as used herein, unless otherwise specified, generally refers to a particle of a relatively small size, but not necessarily in the micron size range. In certain embodiments, microparticles specifically refers to particles having a diameter from about 0.01 to about 500 microns, preferably from about 1 to about 200 microns, more preferably from about 5 to about 25 microns. As used herein, the microparticle encompasses microspheres, microcapsules and microparticles, unless specified otherwise. A microparticle includes a composite construction and is not necessarily a pure substance; it includes a spherical shape or any other shape.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent is dependent on the subject being treated, the severity of the affliction and the manner of administration of the therapeutic composition.

“Treatment” refers to a therapeutic intervention that ameliorates a sign or symptom of a disease or pathological condition after it has begun to develop, or administering a compound or composition to a subject who exhibits only early signs for the purpose of decreasing the risk of developing a pathology or condition, or diminishing the severity of a pathology or condition. The beneficial effect is evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

It is understood that the terminology used in the disclosure is for the purpose of describing particular embodiments and examples only, and is not intended to be limiting.

The invention will now be described, for purposes of explanation, in connection with numerous specific details in order to provide a thorough understanding of the subject innovation. It will be evident, however, that the disclosed concept can be practiced without these specific details without departing from the spirit and scope of this innovation.

The present inventive concepts include the treatment of inflammatory tissue associated with various conditions. For the purpose of ease of description of the invention, the focus is directed to periodontitis that includes sustained release microparticles to locally deliver antibody to reduce inflammation of periodontal tissue. However, it is contemplated and understood that the compositions, methods and applications of the invention are useful for treatment of other tissue inflammation conditions such as, but not limited to, rheumatoid arthritis.

Periodontal tissue (the periodontium) is, by nature, permeated with bacteria. A healthy periodontium depends on an intricate balance between regulators of pro- and anti-immunoinflammatory responses. Cytokines, which are released by different cell types in periodontal tissue, are key regulators of this process and can drive the response either towards destruction or resolution. In this context, the present invention provides approaches to control the host immune response against periodontal pathogens. Without intending to be bound by any particular theory, the systems and methods of the invention consider the destructive and constructive natures of the inflammatory response in tissue, and aim to polarize the immune microenvironment toward a protective and constructive response.

Although the role of IL-17 in the development of periodontal disease can be associated with a protective effect against extracellular pathogens, a direct correlation between increased levels of IL-17 in human gingival crevicular fluid and PD severity has been shown. Accordingly, a destructive role for IL-17 in the pathogenesis of murine PD is considered.

Further, it has been found that the pathogenesis of periodontitis is associated with a high ratio of pro-inflammatory M1 (classically activated) macrophages to anti-inflammatory M2 (alternatively activated).

The inventive concepts include the sustained delivery of anti-IL-17A (IL-17A antibody) as a local therapeutic strategy for murine PD as a disease driven by IL-17 mediated inflammation. The activity of this cytokine is locally modulated by a neutralizing antibody to halt the destruction of periodontal tissues. To this end, poly (lactic-co-glycolic) acid (PLGA) microparticles (MPs) are used to encapsulate the neutralizing antibody and sustainably release IL-17A antibody locally in the periodontium as a therapeutic strategy for murine ligature-induced PD.

In accordance with the inventive concepts, there is developed a local sustained delivery system that aims to restrain IL-17A over-activity in periodontal tissues. A significant decrease in alveolar bone loss has been demonstrated for an anti-IL-17A (IL-17A antibody) incorporated in poly(lactic-co-glycolic) acid microparticles (MP) for local controlled release in the periodontal tissues of mice with ligature-induced PD. Results show that local injection of anti-IL-17A MP 48 hours after PD induction leads to a significant decrease in alveolar bone loss and osteoclastic activity evaluated at 8 days post disease induction. The formulation also shows a decrease in the transcriptional activity of IL-6, a downstream target gene of IL-17A signaling that encodes a cytokine inducer of bone resorption in periodontal tissues. Further, the results demonstrate that a formulation developed to locally and sustainably release anti-IL-17 into the periodontal tissues of mice with PD represents a strategy to control PD progression.

Interleukin-17 (IL-17) is an important player in the pathogenesis of tissue inflammation. IL-17 is primarily produced by a subpopulation of T helper cells called Th17 cells. It is associated with the production of pro-inflammatory cytokines such as IL-6, chemokines and matrix metalloproteinases. This, in turn, leads to host response activation loop, involving innate immune cells, induction of pro-inflammatory signaling pathways and recruitment of neutrophils. In addition, IL-17 induces RANKL production by osteoblasts and other stromal cells, which affect osteoclast-mediated bone resorption. The inventive concepts, taking into account the destructive role of IL-17 on tissue inflammation, modulates the amount of this IL-17 that would be beneficial to decrease the progressive destruction of the tissues. To this end, microspheres composed of a degradable polymer are used to sustainably release IL-17 antibody locally in the tissue, e.g., periodontium. The IL-17 antibody released from the microspheres reduces the effective amount of free IL-17 in the tissue, e.g., periodontal tissue.

In addition to, or in place of, the direct sustainable release of IL-17 antibody directly into inflamed tissue, the inventive concepts include the direct sustainable release of interleukin-4 (IL-4) and/or C-C motif chemokine ligand 2 (CCL2) into inflamed tissue. The microparticle contains, e.g., is loaded with, a degradable polymer and C-C motif chemokine ligand 2 (CCL2), interleukin 4 (IL-4), anti-interleukin 17 (IL17 antibody), or mixtures or blends thereof, and the microparticle is structured to provide a sustained, controlled release of the C-C motif chemokine ligand 2 (CCL2), interleukin 4 (IL-4), anti-interleukin 17, or mixtures or blends thereof, from said degradable polymer to the inflammatory tissue.

A high ratio of pro-inflammatory M1 (classically activated) macrophages to anti-inflammatory M2 (alternatively activated) is associated with the pathogenesis of progressive tissue inflammation. According to the inventive concepts, sustained release, polymer (e.g., PLGA) microparticles locally deliver IL-4 and/or CCL2 to the inflamed tissue to effectively promote inflammation resolution. IL-4 is a T-helper-2 cytokine that induces the polarization of macrophages towards the M2 phenotype by acting through the IL-4 receptor alpha, whereas CCL2 promotes chemotaxis of M0 or M2 phenotype macrophages to the inflamed site and induce their polarization to the anti-inflammatory pro-resolving M2 phenotype. As a result of resolving inflammation, inflammatory bone loss is inhibited and destructive tissue progression, e.g., PD, effectively halted.

According to the inventive concepts, the microparticle systems provide for sustained release of compound, e.g., agent or drug. The agent release is linear or non-linear (a single or multiple burst release). In certain embodiments, the agent is released without a burst effect. For example, the sustained release exhibits a substantially linear rate of release of the therapeutic agent in vivo over a period of time, such as, at least 120 days, or at least 60 days, or at least 20 days. A “substantially linear rate of release”, means that the agent is released at a rate that does not vary by more than about 20% over the desired period of time and, more usually, by not more than about 10%. In certain embodiments, there is a relatively constant rate of release of the agent from the delivery system over the life of the system, e.g., microparticles. The amount of the release varies. For example, the agent is released in amounts from 0.001 to 2 ng per day (i.e., 24 hours) and, more particularly, from 0.1 to 1 ng per day (i.e., 24 hours), for the life of the microparticle system. However, the release rate is increased or decreased depending on the formulation of the microparticle. The desired release rate and agent or drug concentration varies depending on the particular therapeutic agent selected for the agent or drug delivery system, the degree or severity of tissue inflammation, and the subject's health.

The microparticles are fabricated or constructed, e.g., composed or comprised, of polymer. Suitable polymers for use include bio-erodible polymers that are biocompatible. Preferred bio-erodible polymers are polyhydroxy acids, such as, poly lactic acid and copolymers thereof. Illustrative polymers include poly glycolide, poly lactic acid (PLA), and poly (lactic-co-glycolic acid) (PLGA).

Other suitable polymers include, but are not limited to, polyamides, polycarbonates, polyalkylenes, polyvinyl alcohols, polyvinyl ethers, polyvinyl esters, polyvinyl halides, polyvinylpyrrolidones, polyglycolides, polysiloxanes, polyurethanes and copolymers thereof, alkyl cellulose, cellulose ethers, cellulose esters, polymers of acrylic and methacrylic esters, methyl cellulose, ethyl cellulose, poly(methyl methacrylate), poly(ethyl methacrylate), polyethylene, polypropylene polyethylene glycol), poly(ethylene oxide), poly(ethylene terephthalate), poly(vinyl alcohols), poly(vinyl acetate), poly vinyl chloride polystyrene, poly(caprolactone), dextran and chitosan, and mixtures and blends thereof.

In certain embodiments, suitable degradable polymers for use in constructing microparticles in the practice of the inventive concepts include, but are not limited to, PLGA, PLGA copolymer or a blend of PLGA and PLA.

In certain embodiments, the polymer microparticles are biodegradable.

The polymer microparticles are loaded with the compound or agent that includes one or more of IL-4, CCL2 and anti-IL-17A. The polymer microparticles encapsulate the agent. In certain embodiments, the agent-loaded microparticles include a volume average diameter of 10 nm to 500 um and, more particularly, from 5 to 20 um.

The agent-loaded microparticles are pore-less or they contain varying amounts of pores of varying sizes, typically controlled by adding NaCl during the synthesis process.

The compound-loaded (agent-loaded) polymer microparticles are fabricated using various methods. Suitable fabrication methods include single or double emulsion depending on the solubility of the desired encapsulated agent in water, and/or molecular weight of polymer chains used to make the microparticles, which controls the degradation rate of the microparticles and subsequent drug release kinetics.

In certain embodiments, poly lactic-co-glycolic acid (PLGA) microparticles (MP) are fabricated using a conventional water-oil-water double emulsion procedure. An aqueous solution contains anti-mouse IL-17A antibody (e.g., 1 mg/ml) in phosphate buffered saline (PBS), and an oil phase is made of PLGA dissolved organic solvent, such as, dichloromethane. Unloaded MPs (blank MPs) are fabricated according to the same procedure, except that the aqueous solution contains only PBS. The fabricated MPs are then surface characterized using a scanning electron microscope (SEM) and the release profile of the anti-IL-17A antibody (anti-IL-17A) is determined using an enzyme-linked immunosorbent assay (ELISA). The surface characterization of MPs surface morphology is carried out using a scanning electron microscope (SEM). The release profile of the antibody is determined by suspending the anti-IL-17A MPs (e.g., ˜7 mg) in PBS (e.g., 1 ml) and placing the suspension on a tube rotator (e.g., at 37° C.) and collecting supernatants. The amount of anti-IL-17A in the supernatant and its structural integrity is determined using the (ELISA).

In certain embodiments, the molecular weight of the microparticles is from about 1000 Da to 100,000 Da or greater.

The inventive concepts include a pharmaceutical composition that includes loaded microparticles, which include degradable polymer and a compound or agent selected from the group consisting of C-C motif chemokine ligand 2 (CCL2), interleukin 4 (IL-4), anti-interleukin 17 (IL-17 antibody), and mixtures or blends thereof; and at least one ingredient selected from the group consisting of a pharmaceutically acceptable carrier, an adjuvant and an excipient.

The inventive concepts also include a method of administering a therapeutically effective amount of a compound or agent selected from the group consisting of C-C motif chemokine ligand 2 (CCL2), interleukin 4 (IL-4), anti-interleukin 17 (IL-17 antibody), and mixtures or blends thereof, to a subject in need of reduced tissue inflammation, wherein the compound or agent is locally delivered at the site of the tissue inflammation in a sustained release form, and the sustained release form comprises microparticles loaded with the compound or agent.

The inventive concepts also include a method of treating a patient with an inflamed periodontium that includes forming microparticles including degradable polymer and a compound or agent selected from the group consisting of C-C motif chemokine ligand 2 (CCL2), interleukin 4 (IL-4), anti-interleukin 17 (IL-17 antibody), and mixtures or blends thereof, wherein the degradable polymer is loaded with the compound or agent; and administering a therapeutically effective amount of the microparticles locally to the inflamed periodontium, wherein the microparticles are structured to provide a sustained, controlled release of said compound from said degradable polymer to the inflamed periodontium.

In certain embodiments, the methods of the invention involve administering to a subject in need of treatment for tissue inflammation, a pharmaceutical composition that includes a pharmaceutically acceptable carrier and a therapeutically effective amount of the agent-loaded polymer microparticles disclosed herein. The composition or agent-loaded polymer microparticles are administered parenterally including subcutaneous injections or topically. The pharmaceutical composition is administered in dosage unit formulations containing conventional non-toxic pharmaceutically acceptable carriers, adjuvants, and/or vehicles. The loaded microparticles are formulated into suitable pharmaceutical preparations such as solutions or suspensions for parenteral administration.

The loaded microparticles and/or compositions are also provided in kits, for example, including component parts that are assembled for use. In some examples, a kit includes a disclosed loaded microparticle and a second therapeutic compound or agent for co-administration. The therapeutic compound or agent includes one or more of IL-4, CCL2 and anti-IL-17A. Each of the loaded microparticles and therapeutic compound or agent are provided as separate component parts.

The disclosed loaded microparticles or compositions are administered as a single dose, or divided into a number of smaller doses to be administered at intervals of time. The therapeutic compositions are administered in a single dose delivery, by continuous delivery over an extended time period. The loaded microparticles are especially suitable for local and/or sustained delivery. For local delivery, the loaded microparticles are applied directly to the tissue or organ for which treatment is sought. The effect of local delivery is limited primarily to the tissue or organ to which the loaded microparticles are applied. In some embodiments, the pharmaceutical composition is formulated for injection containing the loaded mircroparticles and a pharmaceutical excipient suitable for injection. Sterile injectable solutions are prepared by incorporating the loaded microparticles in the required amount in the appropriate solvent with various other conventional ingredients.

The loaded microparticles are effective, e.g., they work, in the following three separate clinical scenarios:

(i) when the tissue is healthy, and disease is induced but microparticles are also delivered, the disease is prevented from occurring (“preventative”);

(ii) when the tissue is inflamed (disease is in progress), then the microparticles are injected, and the disease is halted or stopped (“interventional”); and

(iii) when the cause of the disease is removed (remove the ligature), then microparticles are injected, and healing and regeneration at the site is enhanced.

In the claims, any reference signs placed between parentheses shall not be construed as limiting the claim. The word “comprising” or “including” does not exclude the presence of elements or steps other than those listed in a claim. The word “a” or “an” preceding an element does not exclude the presence of a plurality of such elements. The mere fact that certain elements are recited in mutually different dependent claims does not indicate that these elements cannot be used in combination.

### EXAMPLES

In the following examples, the histological analysis was conducted as follows. The fixed maxillae were demineralized in 10% EDTA for 15 days at 4° C. then processed for histological analysis. Paraffin embedded hemi-maxillae were sectioned in serial 4 μm thick sections in a buccal-palatal direction. Sections were stained for tartrate resistant acid phosphatase (TRAP; Sigma-Aldrich, Saint Louis, Mo., USA) and counterstained with hematoxylin for evaluation of osteoclasts quantification. Only the sections in which the pulp chamber and the two roots of the 2^{nd} molar could be visualized were selected for further analysis. For each sample, four maxillae sections, at a distance of at least 16 μm intervals, were utilized for identifying and counting of osteclasts at 400× magnification. In selected sections, the coronal two thirds of the mesial and distal alveolar bone crest adjacent to the maxillary 2^{nd} molar and furcation area was demarcated using Image J software as regions of interest (ROIs). Osteoclasts were identified as TRAP-positive, multinucleated cells with purple cytoplasm situated on the marginal alveolar bone of the demarcated areas. Results were presented as the average number of osteoclasts divided by the surface are of the ROIs in mm² in each experimental group.

In the following examples, the quantitative polymerase chain reaction (qPCR) was conducted as follows. The frozen maxillae samples were first crushed in liquid nitrogen then RNA extraction was performed using RNeasy Mini Kit (Qiagen, Valencia, Calif.). The gene expression of interleukin-17A (IL-17A), interleukin-6 (IL-6), tumor necrosis factor alpha (TNF-α), and receptor activator of nuclear factor kappa-B ligand (RANKL) was assessed by qPCR using TaqMan probes (Applied Biosystems, Foster City, Calif.). Real-time PCR was performed using QuantStudio™ 6 Flex Real-Time PCR system (Thermo Fisher Scientific). Data were analyzed using the delta-delta Ct method and presented as the mean fold change normalized to the healthy control group.

### Example 1

There was a local injection of IL-17Ab MP 48 hours after PD induction. Eight days post disease induction, alveolar bone loss was evaluated. Results suggest that the local injection of IL-17Ab MP 48 hours after PD induction led to a significant decrease in alveolar bone loss, as evaluated eight days post disease induction. The formulation also decreased the number of osteoclasts in alveolar bone and the levels of IL-6, a cytokine related to bone resorption, in periodontal tissues. In conclusion, it was demonstrated that a formulation developed to sustainably release IL-17 Ab directly into the periodontal tissues of mice with PD represents a strategy to manage PD.

### Material and Methods

### Microparticles (MP) Fabrication and Characterization

Poly lactic-co-glycolic acid (PLGA) microparticles (MPs), loaded with the anti-IL-17A antibody, were fabricated using a standard water-oil-water double emulsion procedure. The aqueous solution contained anti-mouse IL-17A antibody (1 mg/ml) in phosphate buffered saline (PBS), while the oil phase was made of PLGA dissolved in dichloromethane as on organic solvent. Unloaded MPs (blank MPs) were fabricated following the same procedure, except that the aqueous solution contained only PBS. The fabricated MPs were then surface characterized using a scanning electron microscope (SEM) and the release profile of the anti-IL-17A antibody (anti-IL-17A) was determined over 2 weeks using an enzyme-linked immunosorbent assay (ELISA).

More particularly, 200 μl of an aqueous solution containing the anti-IL-17A antibody (anti-mouse IL-17A; 1 mg/ml, Affymetrix eBioscience) in phosphate buffered saline (PBS) (Thermo Fisher Scientific, Waltham, Mass.) was added to 200 mg PLGA (719897-5G; Sigma-Aldrich, St. Louis, Mo.) dissolved in 4 ml dichloromethane (Sigma-Aldrich). The resultant mixture was sonicated for 10 s (first emulsion of water-in oil) then added to 60 ml of 2% poly (vinyl alcohol, PVA) solution (M.W.=25 000 gmol-1, 98 mol % hydrolyzed, Ploy Sciences, Warrington, Pa.), and then homogenized (FSH125; Fisher Scientific) for 1 minute at 3000 rpm. The homogenized solution (second emulsion) was then added to 80 ml of 1% PVA and stirred for 3 hours to allow for evaporation of dichloromethane. Next, the MPs in suspension were centrifuged and washed 4 times in deionized (DI) water. The MPs were then re-suspended in DI water (5 ml), flash frozen in liquid nitrogen and lyophilized using LABCONCO FREEZONE 4.5 freeze dryer (Labconco, Kansas City, Mo.) for 72 h. Unloaded MPs (Blank MPs) were fabricated following the same procedure, except that the aqueous solution contained only PBS.

The surface characterization of MPs surface morphology was carried out using a scanning electron microscope (SEM) (JSM-6330F; JEOL). The release profile of the antibody was determined by suspending the anti-IL-17A MPs (˜7 mg) in 1 ml of PBS, and placing the suspension on a tube rotator at 37° C. and collecting supernatants daily for two weeks. The amount of anti-IL-17A in the supernatant and its structural integrity was determined using an enzyme-linked immunosorbent assay (ELISA).

### MP Characterization

MPs were characterized by their shape and the release profile of the IL-17 antibody (IL-17Ab). MP surface morphology was characterized using a scanning electron microscope (SEM) (JSM-6330F; JEOL). In vitro release characteristics were determined by suspending IL-17AbMP (˜7 mg) in 1 ml of PBS and exposing to end-over-end rotation at 37° C. for two weeks. The suspensions were centrifuged daily, with the supernatant being collected and the IL-17AbMP pellet re-suspended in 1 ml of PBS at different time points. The amount of IL-17 Ab in the supernatant was determined using an enzyme-linked immunosorbent assay (ELISA). To determine IL-17 Ab loading, the same amount of IL-17AbMP (7 mg) formulation was solubilized in 0.05 N NaOH+0.5% sodium dodecyl sulfate (SDS) solution (10 ml) for 2 h, followed by quantification of the amount of antibody in the supernatant via BCA Assay (Thermo Fisher Scientific, Waltham, Mass.).

### Murine Ligature-Induced Periodontal Disease

Six to eight weeks old male BALB/c mice (Jackson Laboratory, Bar Harbor, Me.) were used in this study. Mice were maintained under a 12/12 h light/dark cycle at 23-25° C. with free access to water and commercial food. The study was approved by Institutional Animal Care and Use Committee (IACUC) of the University of Pittsburgh.

The murine ligature PD model employed in this study was previously described. Briefly, mice were anaesthetized with a mixture of Ketamine (80 mg/kg) and Xylazine (8 mg/kg), then a sterile 6-0 silk suture (Henry Schein®) was ligated around the maxillary second molar at the level of the gingival sulcus to induce plaque accumulation. The contralateral non-ligated maxillary molar served as an internal control.

### Experimental Design, Anti-IL-17A MPs Local Delivery and Samples Collection

After ligature placement, the animals were divided into three experimental groups: (1) Untreated: ligature placement with no MPs injection, (2) anti-IL-17A MPs—day 0: ligature placement with Anti-IL-17A MPs injection on the same day and (3) IL-17A MPs—day 2: ligature placement with Anti-IL-17A MPs injection after 48 hours. All mice were sacrificed on the 8^{th} day after ligation (n=5 mice).

For the second and third groups, anti-IL-17A MPs suspended in PBS with 2% carboxymethylcelulose (25 mg/ml) were locally delivered using a 28.5-gauge insulin syringe into four different sites (10-15 ul/site) in the buccal and palatal gingiva surrounding the ligated tooth under a stereomicroscope.

To rule out the possibility that PLGA may exert an effect on bone loss, an independent experiment was conducted where alveolar bone loss was compared between mice with ligature placement only (no MPs injection) and mice with ligature and local injection of unloaded (blank) MPs (Supplementary FIGS. 1A and 1B).

At the end of the experiment, mice were euthanized and the maxillae were harvested and placed in either 10% Formaldehyde for fixation then alveolar bone loss and histological analysis, or in liquid nitrogen followed by storage at 80° C. for RNA extraction and qPCR analysis.

### Alveolar Bone Loss Quantification

To quantify alveolar bone loss, the fixed mice maxillae, were transferred to 70% ethanol for micro-computed tomography (μCT) scanning using a viva CT 40 microCT system (SCANCO Medical, Brüttisellen, Switzerland) at a resolution of 10.5 μm and 55 μA kVp. The scans were reoriented in a standardized manner by Data Viewer software (GE Healthcare) using fixed anatomical landmarks to allow reproducible evaluation of bone loss across all samples. Bone loss was determined by measuring the distance between the cementoenamel junction (CEJ) and the alveolar bone crest (ABC) on equidistant scanning slices on both the sagittal and coronal views.

More particularly, the distance between the cementoenamel junction (CEJ) and alveolar bone crest (ABC) was measured in the sagittal and coronal planes. Scans were oriented so that a horizontal line crossed the CEJ of the first and second molars in the sagittal plane, while a vertical line crossed the center of the pulp chamber of all molars in the transaxial plane. After reorientation, linear bone resorption was assessed by measuring the distance between the CEJ and the ABC using Image J software. On the buccal and palatal aspects, the ABC-CEJ distance was measured in 47 coronal sections spanning all molars, with 52.5 μm intervals in between successive sections. On the interdental aspects, bone loss was assessed in 11 sagittal sections, with 52.5 μm intervals in between, on both the mesial and distal sides of the second molar. The interdental bone loss was calculated as the average of mesial and distal measurements. Measurements were performed on both the ligated (experimental) and non-ligated (healthy) sides of the maxillae. For each aspect, data were presented as the differences between the average values on the ligated and non-ligated sides. The investigator conducting the measurements was blinded from the treatment received in the experimental group being analyzed.

### Histological Analysis

The fixed maxillae were demineralized in 10% EDTA for 15 days at 4° C. and processed for paraffin embedding histological analysis. Paraffin embedded half-maxillae were sectioned in serial 4 μm thick sections in a buccal-palatal direction. Sections were stained for tartrate resistant acid phosphatase (TRAP; Sigma-Aldrich, Saint Louis, Mo., USA) and counterstained with hematoxylin for evaluation of osteoclastic activity.

More particularly, only the sections in which the pulp chamber and the two roots of the 2^{nd} molar could be visualized were selected for further analysis. For each sample, four maxillae sections, at a distance of at least 16 gm intervals, were utilized for identifying and counting of osteclasts at 400× magnification. In selected sections, the coronal two thirds of the mesial and distal alveolar bone crest adjacent to the maxillary 2^{nd} molar and furcation area was demarcated using Image J software as regions of interest (ROIs). Osteoclasts were identified as TRAP-positive, multinucleated cells with purple cytoplasm situated on the marginal alveolar bone of the demarcated areas. Results were presented as the average number of osteoclasts divided by the surface are of the ROIs in mm² in each experimental group.

### TRAP Staining

The left and right maxillae halves were excised, fixed in 10% buffered formalin solution pH 7.2 for 48 h, demineralized in 10% EDTA for 15 days at 4° C., and processed for standard histological analysis. Paraffin blocks were cut in serial 4 μm sections in a buccal-palatal direction, stained for tartrate resistant acid phosphatase (TRAP; Sigma-Aldrich, Saint Louis, Mo., USA) and counterstained with hematoxylin. Only the sections in which the pulp chamber and the two roots of the 2^{nd} molar could be visualized were selected for further analysis. For each sample, four maxillae sections, at a distance of at least 16 μm intervals, were analyzed (400×). In selected sections, the coronal two thirds of the mesial and distal alveolar bone crest adjacent to the maxillary 2^{nd} molar and furcation area was delimited using Image J software. Osteoclasts were identified as TRAP-positive, multinucleated cells situated on the marginal alveolar bone of the delimited areas. Results are presented as the average number of osteoclasts/mm² in each group.

Quantitative Polymerase Chain Reaction (qPCR)

The frozen maxillae samples were first crushed in liquid nitrogen then RNA extraction was performed using RNeasy Mini Kit (Qiagen, Valencia, Calif.). The gene expression of interleukin-17A (IL-17A), interleukin-6 (IL-6), tumor necrosis factor alpha (TNF-a), and receptor activator of nuclear factor kappa-B ligand (RANKL) was assessed by qPCR using TaqMan probes (Applied Biosystems, Foster City, Calif.). Real-time PCR was performed using QuantStudio™ 6 Flex Real-Time PCR system (Thermo Fisher Scientific). Data were analyzed using the delta-delta Ct method and presented as the mean fold change normalized to the healthy control group.

### Statistical Analysis

Results were presented as means±SD of 5-6 mice per group. Differences between means were evaluated using one-way ANOVA followed by Tukey (bone loss and osteoclastic activity) or Holm-Sidak (gene expression) post-hoc multiplecomparison tests. P-value of less than 5% (p<0.05) was considered statistically significant. Statistics were performed using GraphPad Prism.

### Results

Sustained Release Profile of a Functionally Active Anti-IL-17 A from PLGA MPs

The anti-IL-17A MPs supernatants collected daily were used to assess the release profile of anti-IL-17A encapsulated in PLGA by ELISA. The release profile of anti-IL-17A from the PLGA MPs showed an initial burst release of 0.7 ng/ml, followed by a relatively steady release of additional 0.1 ng/ml per day, continuing until day 11. An increase in the anti-IL-17A release rate took place starting day 12 with 0.2 ng/ml being released until day 14 (FIG. 1A). Total loading of anti-IL-17A in PLGA was 20% of the amount initially used in the fabrication. SEM images revealed spherically shaped non-porous anti-IL-17A MPs with an average diameter of 17.8 μm, which is within the known effective size range for PLGA MP intended for drug delivery (FIGS. 1b and 1c). Although PLGA MP smaller than 10 μm are more susceptible to phagocytosis by immune cells, MP larger than 10 μm will remain in the extracellular matrix tissue releasing encapsulated protein locally at the site of the depot.

The ELISA results confirm not only the quantity of IL-17A Abs released from the PLGA MP, but also that the structural integrity of the ABS was maintained given that this assay is based on the binding of functionally active IL-17A antibody to its epitope on IL-17A. Thus, it is likely that the released IL-17A Abs will exert neutralizing activity upon local delivery.

### IL-17A MPs Inhibited Alveolar Bone Loss and Reduced Osteoclast Count in PD

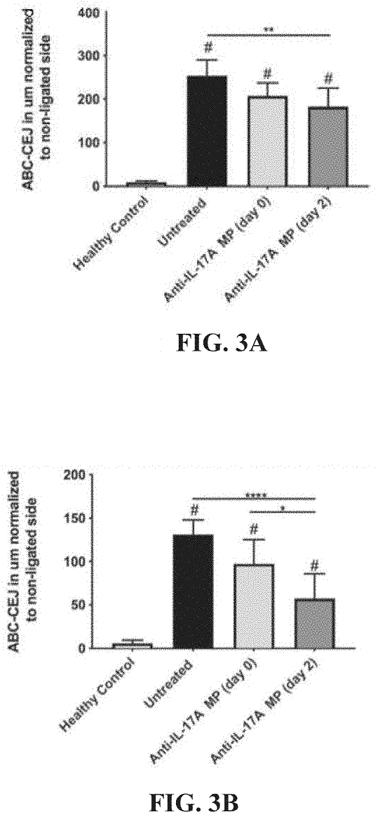

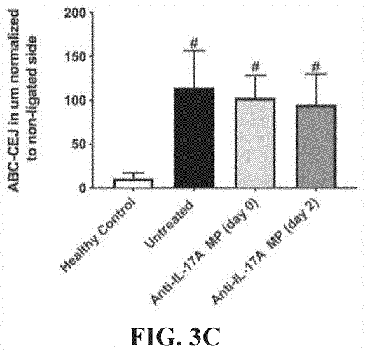

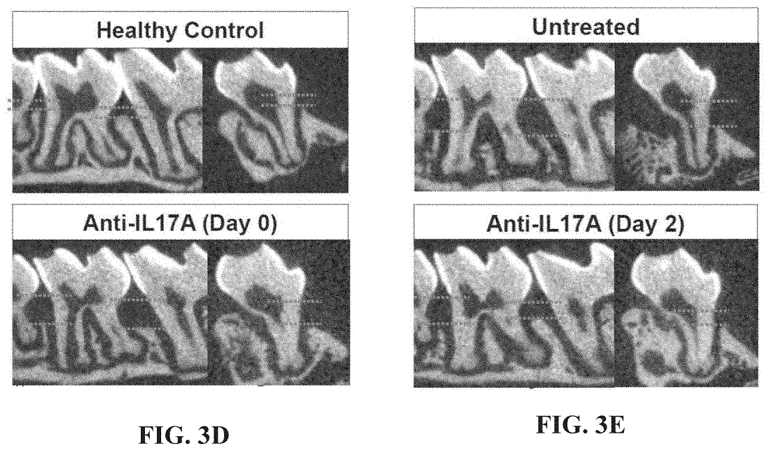

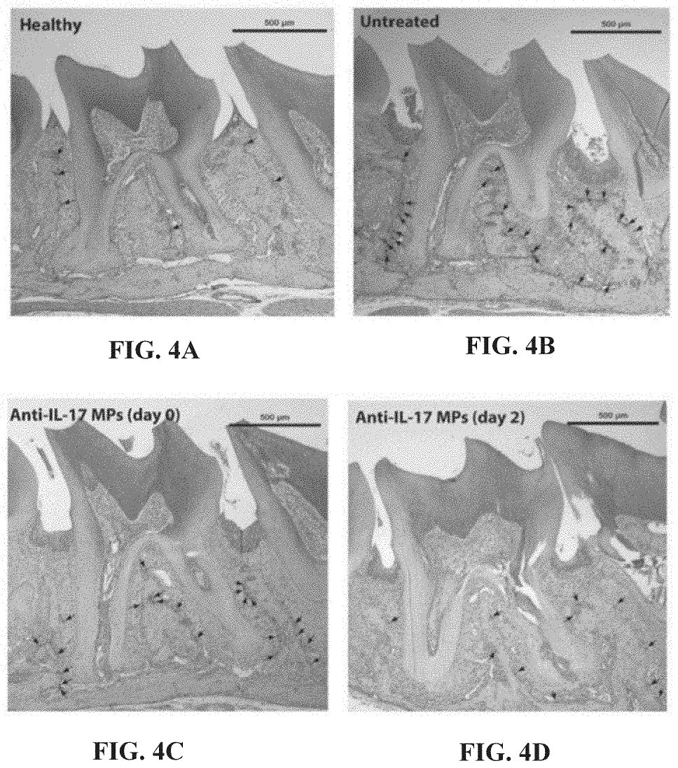

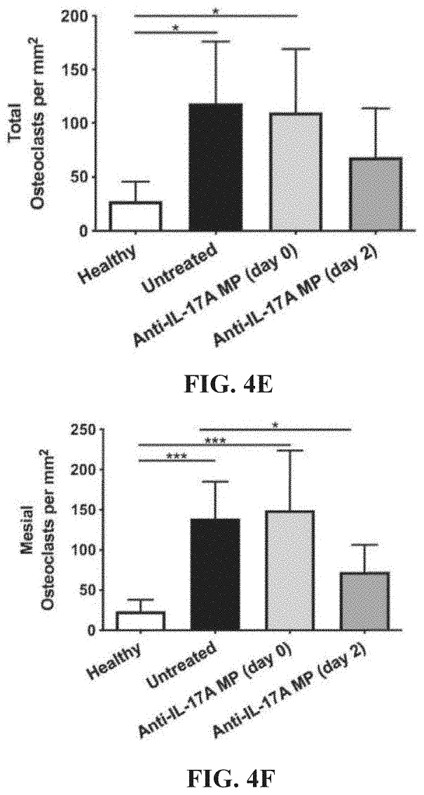

To determine the impact of local sustained delivery of anti-IL17A on murine periodontal bone loss, a standard ligature-induced mouse model of PD was employed. Anti-IL-17A MPs were administered contemporaneously with (Anti-IL-17A D0) or 2 days after (Anti-IL-17A D2) the start of PD induction by ligature. Compared to untreated controls, the anti-IL-17A MP D2 group showed inhibition of alveolar bone loss on the buccal and interdental aspects of the ligated molar as determined by μCT analysis (FIGS. 3A, 3B and 3C). Additionally, the osteoclast count in the anti-IL-17A MP D2 group was lower than the untreated group on the mesial aspect of the ligated molar as determined by TRAP staining (FIGS. 4A-4F). Administration of unloaded PLGA MP did not alter the extent of bone loss or osteoclastic activity compared to the untreated/ligature control group (Supplementary FIGS. 1A and 1B), confirming that the PLGA delivery system does not impact alveolar bone loss in ligature PD.

### IL-17A Expression in Murine PD and Anti-IL-17A MPs Local Delivery Effect on IL-6

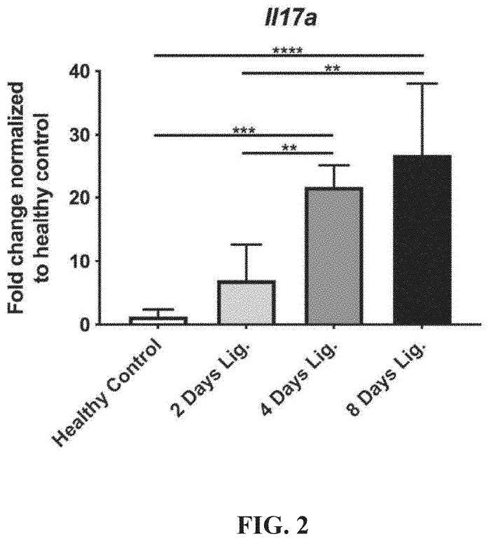

To determine the expression profile of IL-17A and its dependent genes during ligature induced PD, RNA was extracted from periodontal tissues at different time point days following ligature placement (FIG. 2). Upregulation of IL-17A mRNA expression was observed starting at day 2 post-ligature placement, evident at day 4 and peaking at day 8. The gradually increasing expression profile of IL-17A during murine ligature induced PD promoted the delivery of the anti-IL-17A MPs 2 days after, instead of concurrently with, placing ligatures for all subsequent experiments.

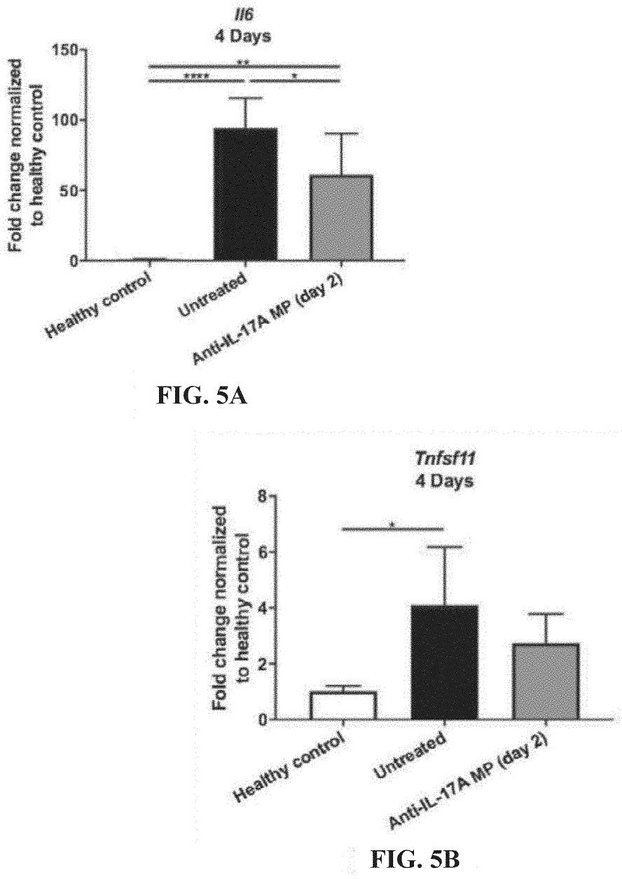

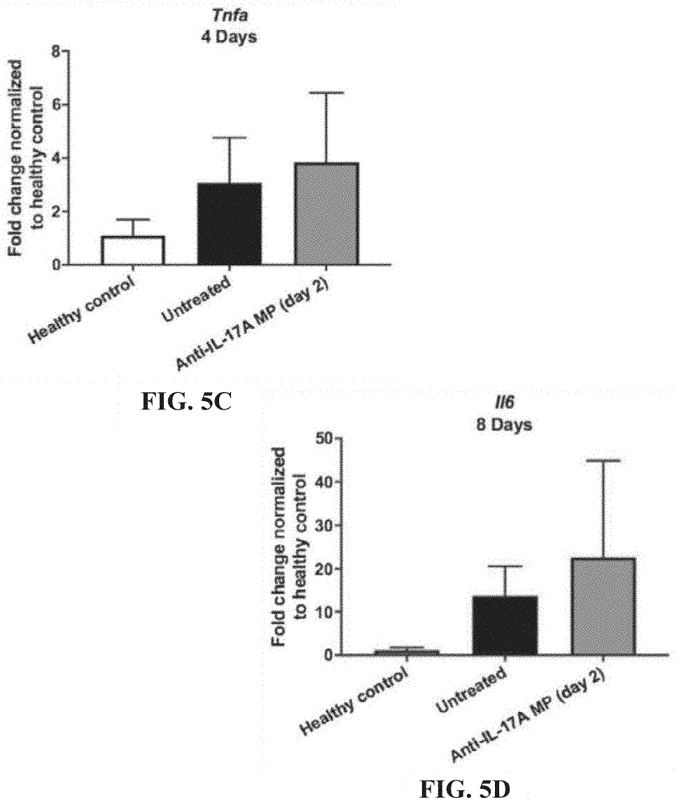

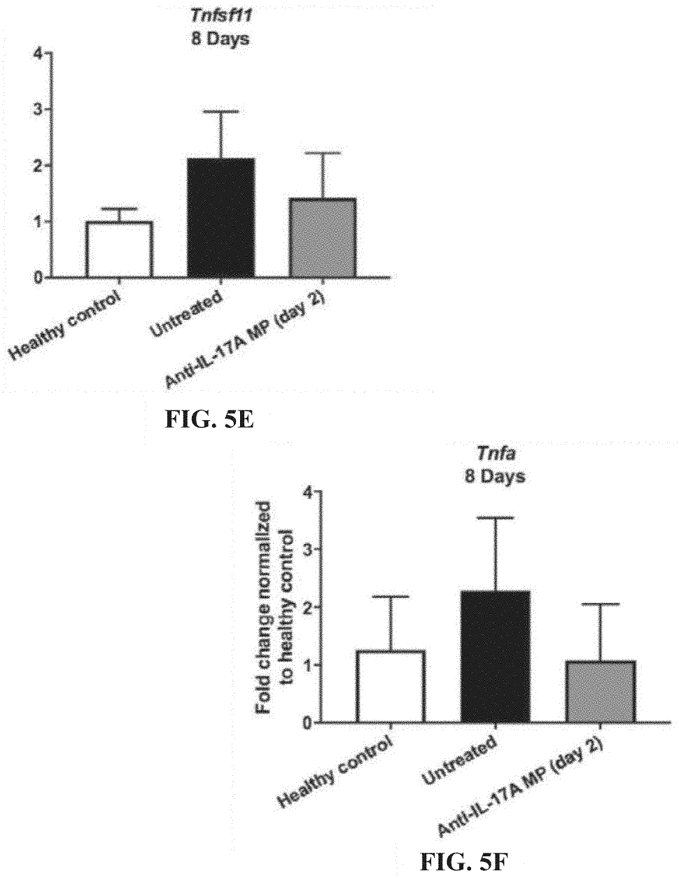

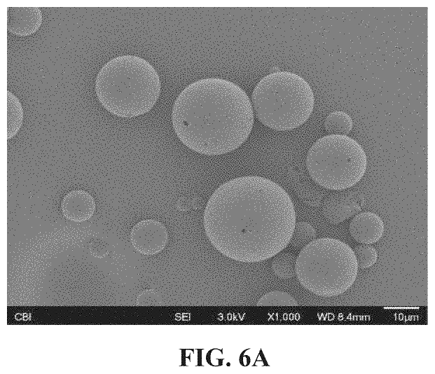

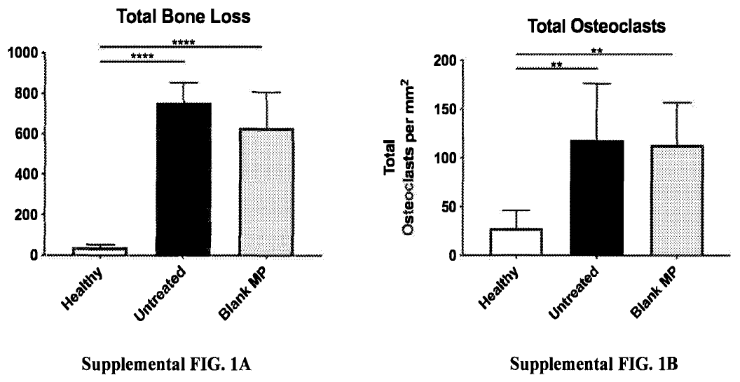

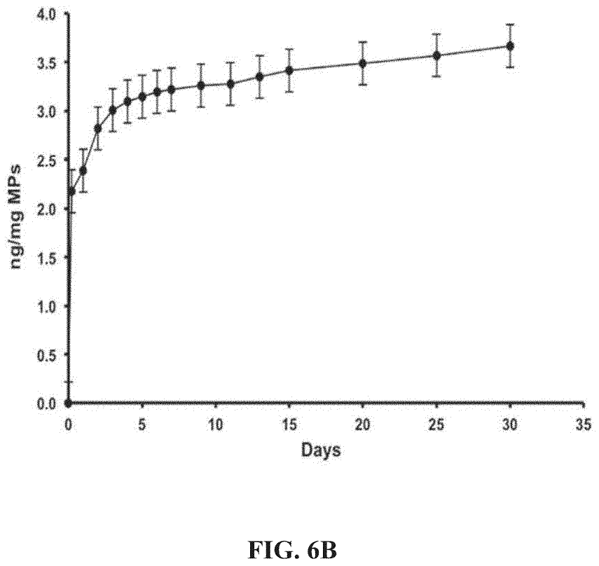

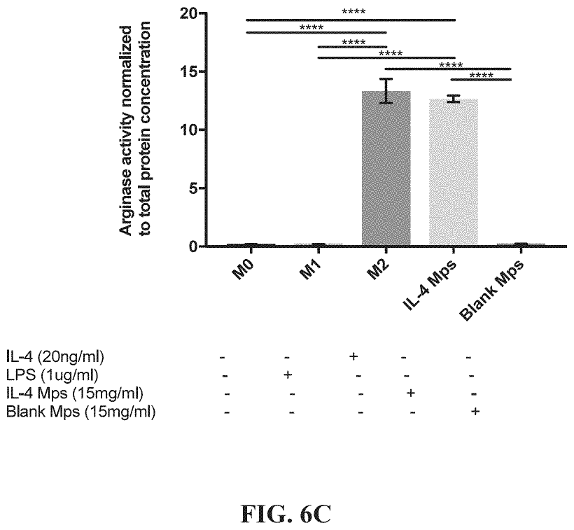

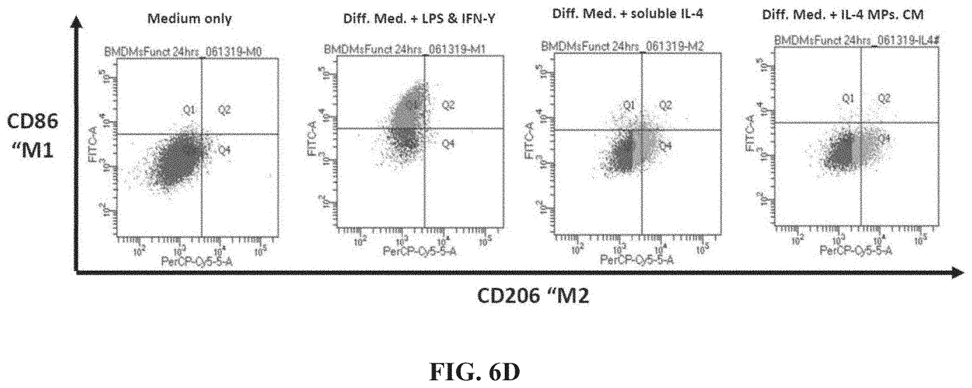

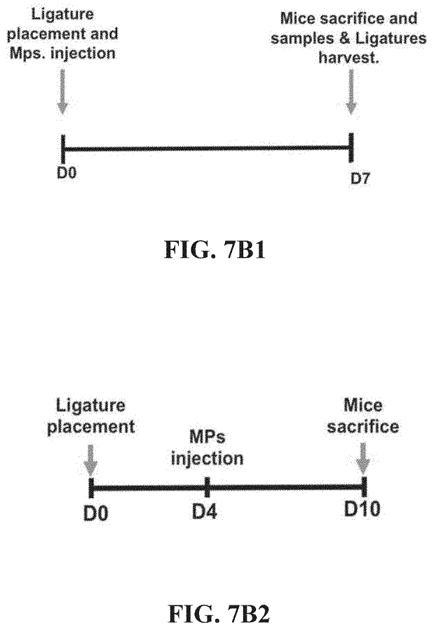

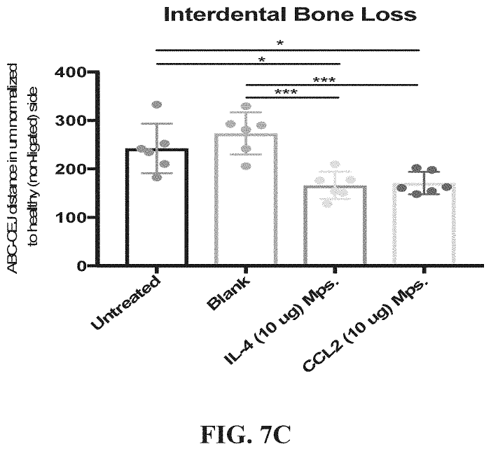

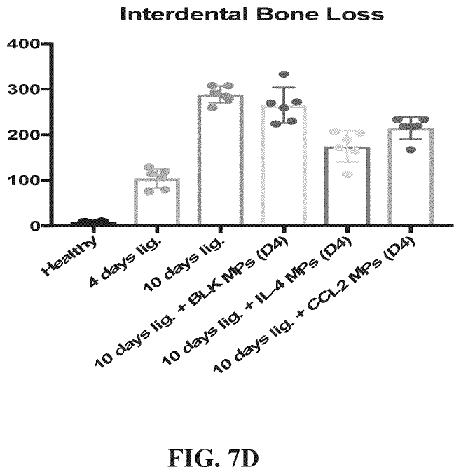

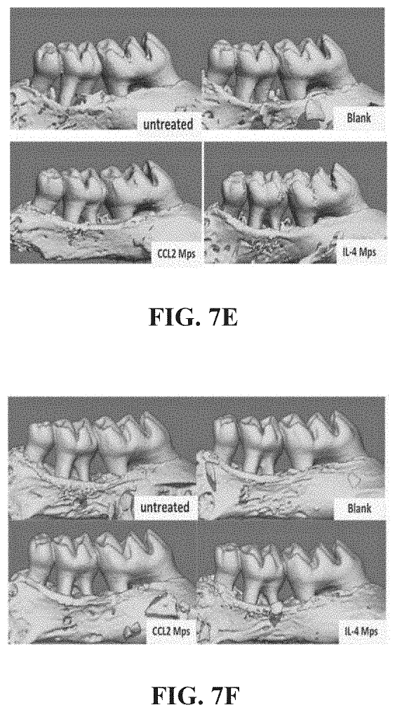

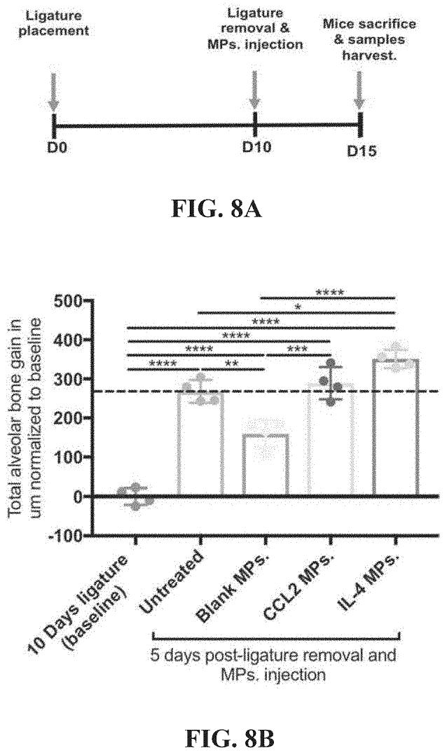

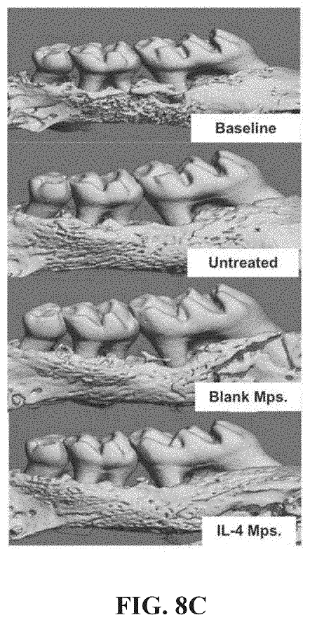

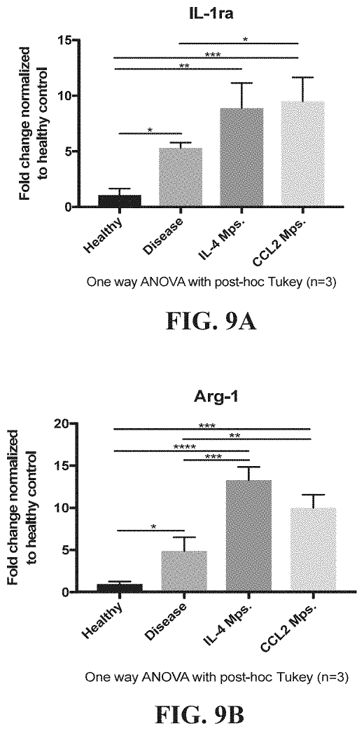

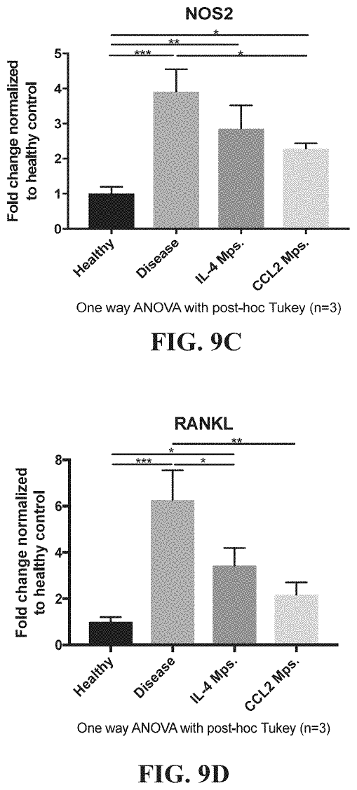

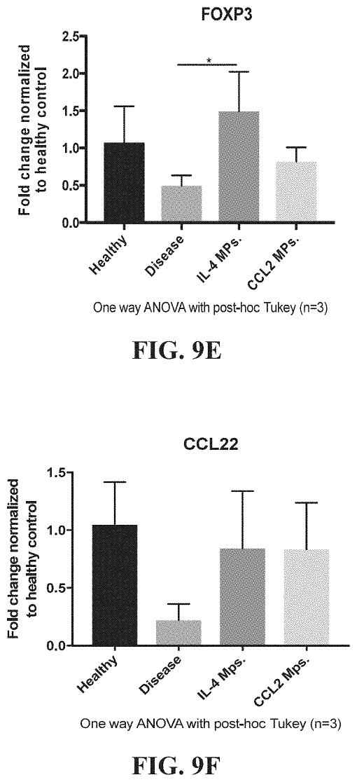

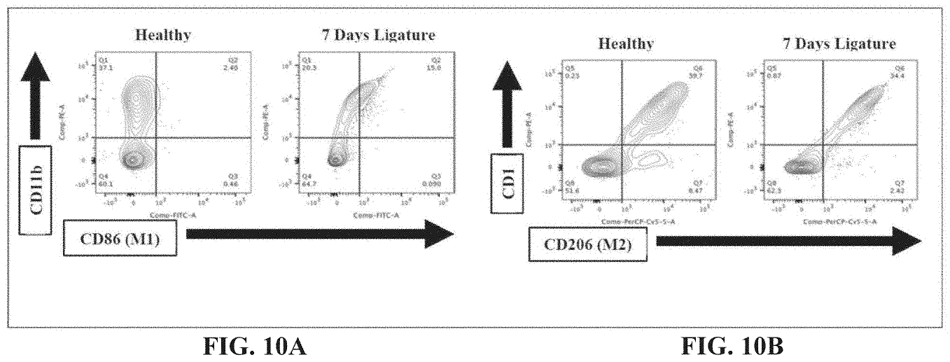

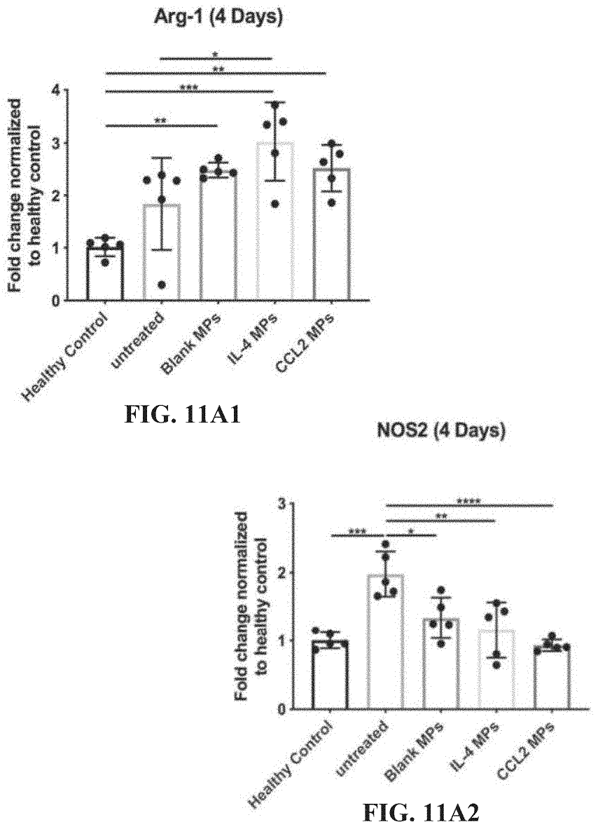

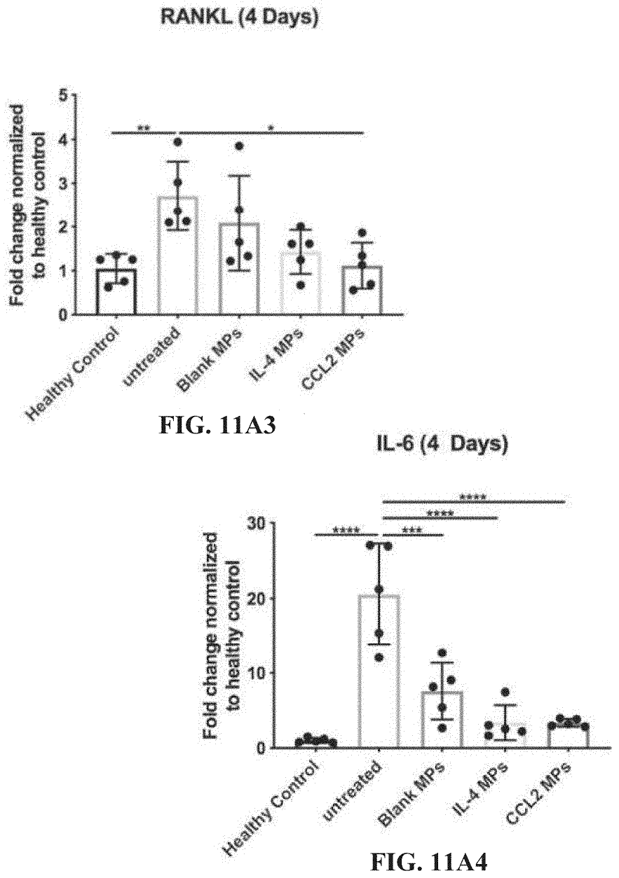

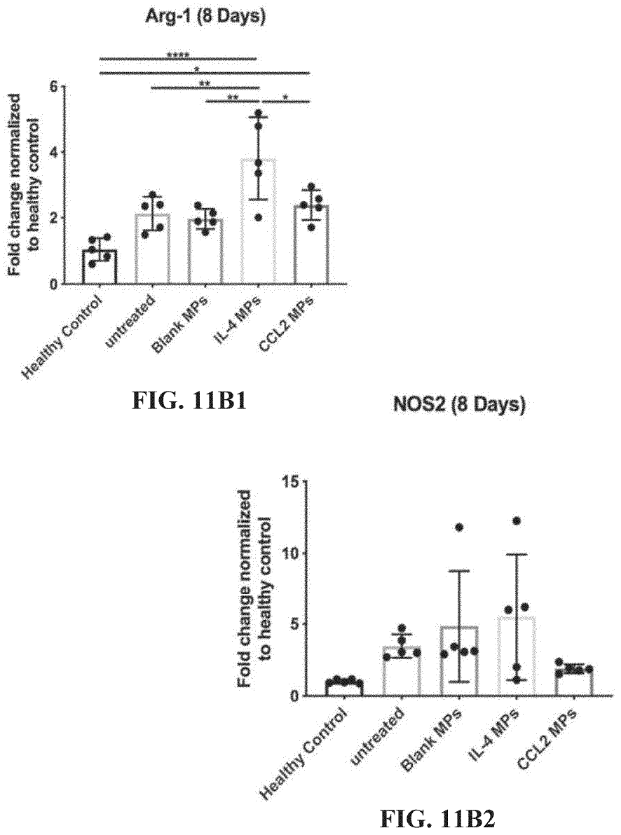

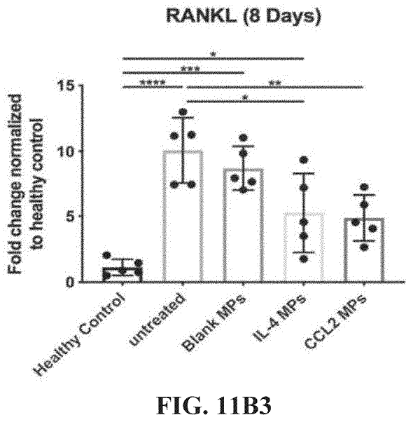

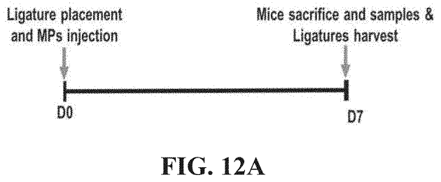

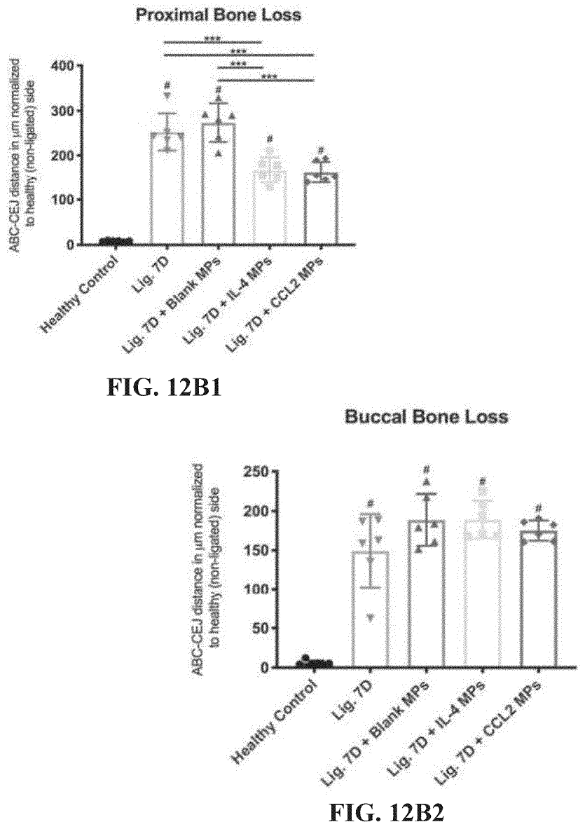

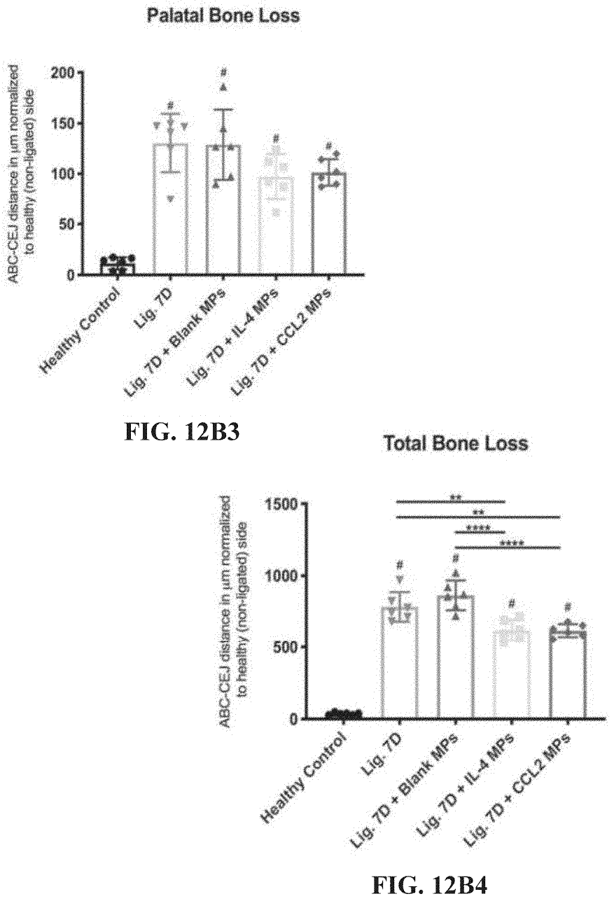

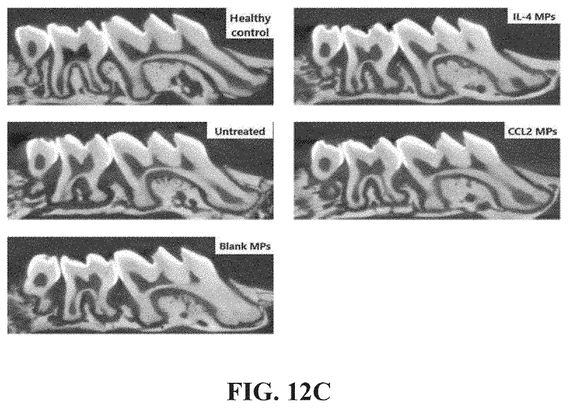

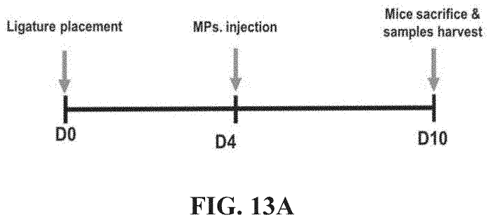

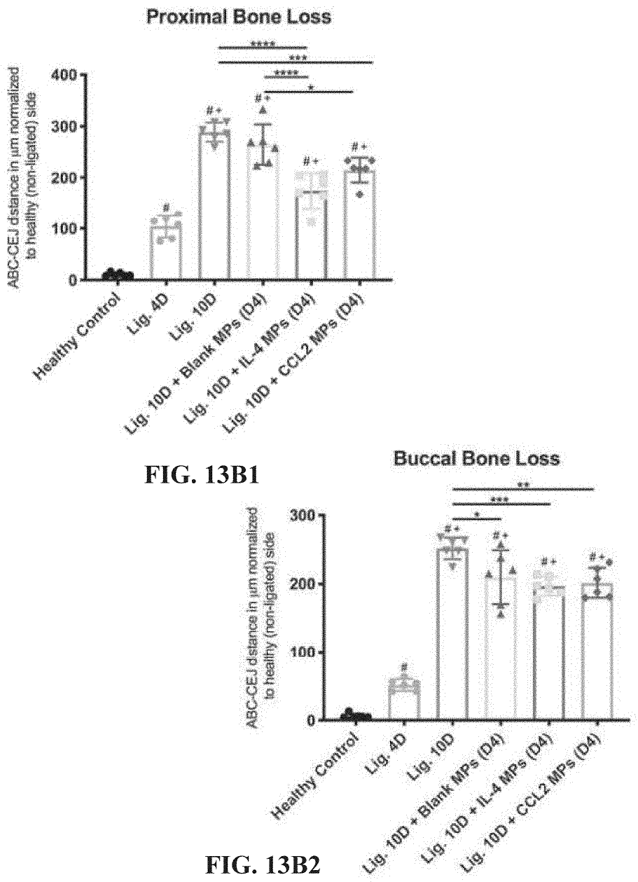

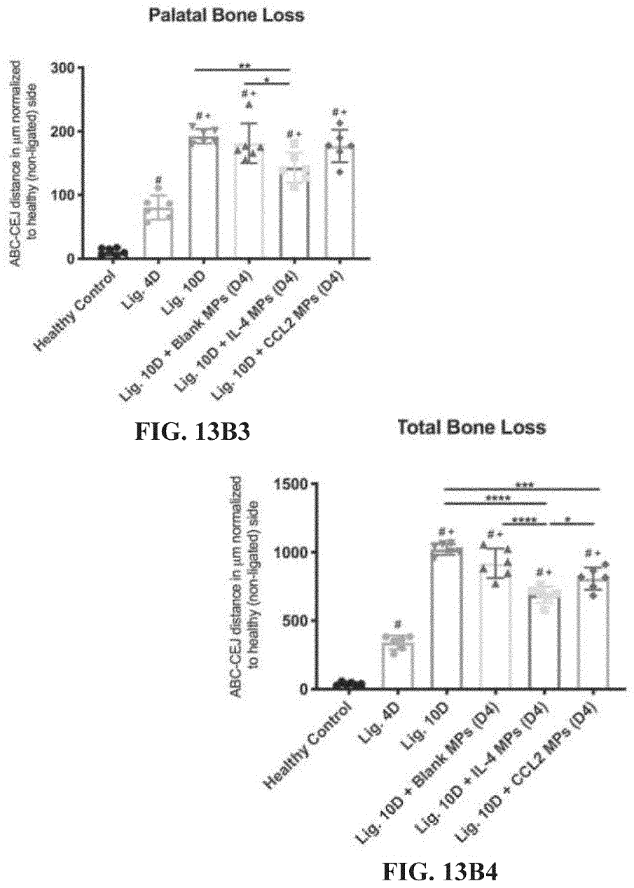

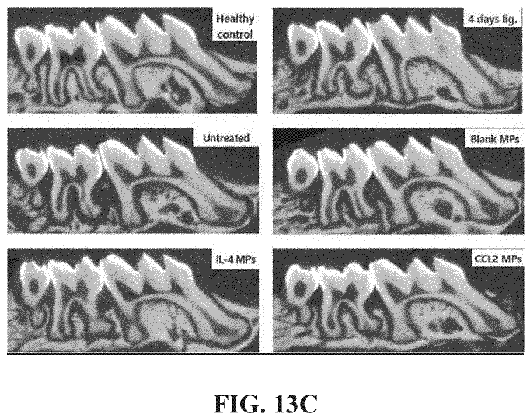

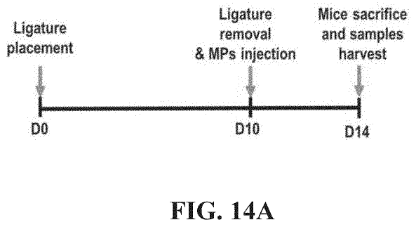

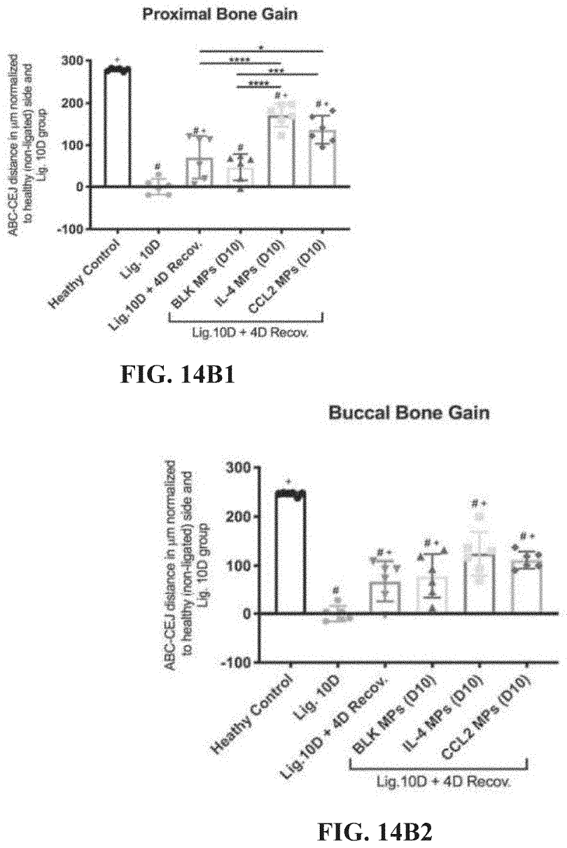

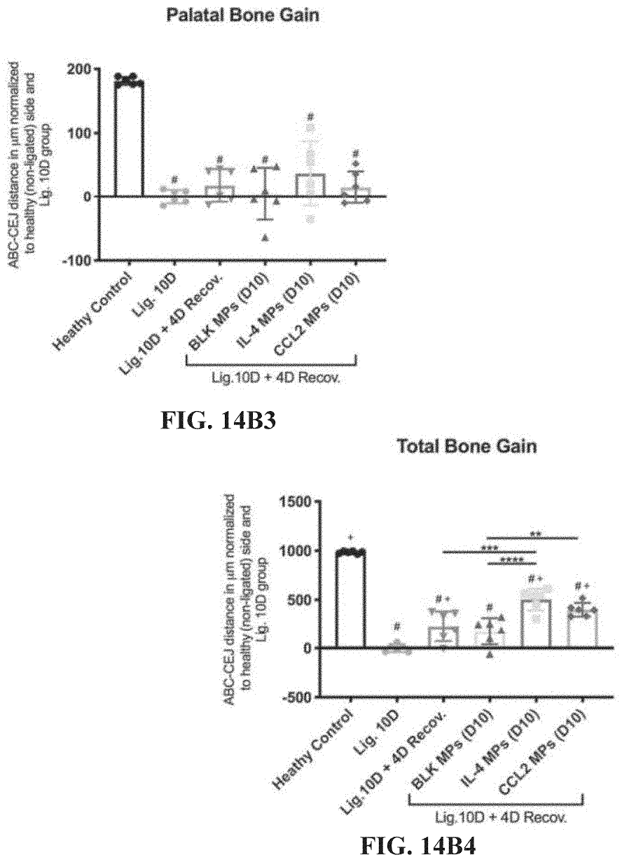

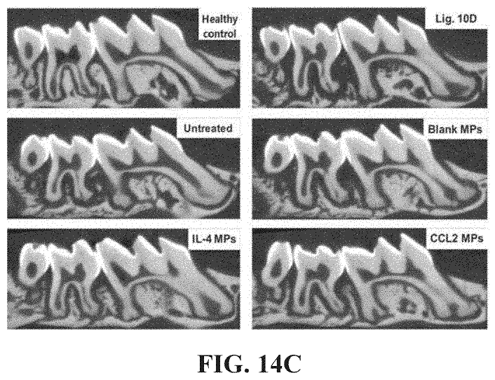

Expression of Il6, Tnfa, and Tnfsf11 (RANKL) mRNA in periodontium was evaluated at either day 4 or day 8 after ligature placement. At day 4, the untreated/ligature-only group showed elevated Il6 and Tnfsf11 compared to controls. Anti-IL-17A MP administration reversed upregulation of Il6 but not Tnfsf11 (FIGS. 5A and 5C). Tnfa was not different among experimental groups (FIG. 5B). At day 8, Tnfsf11 showed sustained upregulation in the untreated group compared to controls. Mice given anti-IL-17A MPs exhibited no differences in the expression of Il-6, Tnfa, and Tnfsf11 mRNA compared to the heathy controls or the untreated groups at day 8 (FIGS. 5D, 5E and 5F). Collectively, these data show that local neutralization of IL-17A in diseased periodontium inhibits Il6 mRNA expression, recapitulating the previously reported effect of IL-17A on Il6 expression.

### Discussion

A healthy periodontium depends on an intricate balance between the host response and the periodontal microbiota. In PD, changes in the periodontal microbial load and/or composition (dysbiosis) disturbs the host-microbe homeostasis and provokes inflammation-driven tissue destruction. Because periodontal damage is primarily caused by an uncontrolled host inflammatory response to a dysbiotic microbiota, several groups have investigated host modulation as a therapeutic approach for PD. In this regard, several approaches based on dampening specific pro-inflammatory mediators that drive inflammatory bone loss have been investigated. The inventors have demonstrated that recruitment of regulatory T lymphocytes or induction of pro-resolving M2 macrophages in murine periodontium protected against inflammatory bone loss.

IL-17A is the signature cytokine of Th17 cells, as well as innate lymphocytes cells such as γδ-T cells, NKT and ILC3s. In the present study, there was evaluated the therapeutic efficacy of modulating IL-17A in murine ligature PD as an exemplar for chronic osteolytic diseases. The goal was to provide a proof of concept for the application of this therapeutic strategy to ameliorate bone destructive diseases involving IL-17A. To that end, a formulation was developed that sustainably releases functionally-active IL-17A Abs that can suppress the destructive effects of IL-17A in PD locally over a period of two weeks (FIG. 1).

In the present study, there was observed an inhibition in alveolar bone loss only when anti-IL-17A MPs were delivered 2 days after, but not simultaneously with, PD induction. One possible explanation for this observation is the difference in timing between the secretion of IL-17A in the inflamed periodontium and the release of the IL-17A Abs from the MP (FIG. 1A). The qPCR data showed that IL17a mRNA expression in murine periodontal tissues following PD induction is modest at day 2 then sharply increases at day 4, remaining elevated up to day 8 (FIG. 2). It was speculated that the rise in Il17a from day 2 to day 4 marks the emergence of a pathogenic, rather than homeostatic, subset of IL-17A-producing cells in the inflamed periodontal environment, denoting a switch from a protective to a destructive IL-17A driven inflammation. To that end, the experiment was designed so that the anti-IL-17A burst release takes place just before the emergence of pathogenic IL-17A-producing cells. This temporal synchrony between the anti-IL-17A burst release and the hypothesized window for IL-17A hyperactivity may explain the protective effect of delivering anti-IL-17A MP 2 days after ligature placement and not earlier.

The IL-17A targeted approach herein is of potential clinical significance since IL-17A-producing cells present at periodontal sites are not always pathogenic in nature. On one hand, one study suggested that a homeostatic subset of Th17 cells arise in the gingiva as a result of physiological forces of mastication, and this population is essential for immune surveillance and host defense at oral this oral barrier sites. On the other hand, a pathogenic Th17 cell subset expands locally in response to microbial dysbiosis and drives inflammatory damage in murine ligature PD. The latter suggests that the destructive inflammatory response in murine PD is induced by IL-17A-dependent signaling. Thus, it is evident that the effects of IL-17A and its producing cells in tissue can be context dependent. Therefore, an understanding of IL-17A dynamic and divergent functions should form the basis for deploying IL-17A targeted therapies in a specific clinical setting.

Osteoclastic bone resorption is a hallmark of PD and is regulated by diverse signals within the bone microenvironment. In line with the findings of alveolar bone loss suppression, there was observed a reduction in the number of osteoclasts in the alveolar bone crest area in the group that received local anti-IL-17A MP 2 days after ligature placement. Indeed, IL-17A can upregulate the pro-osteoclastogenic cytokines IL-6 and IL-8, either alone or in synergy with other cytokines such as IL-1β and TNF-α (Noack et al. 2019). The synergistic interaction between IL-17A and TNF-α was suggested as a predictor of early joint damage in RA patients. Similarly, the synergism between IL-17A and IL-1β enhanced bone destruction in explants from RA patients. Therefore, blockade of IL-17A may indirectly inhibit the effects of other inflammatory cytokines by mitigating their synergistic interactions.

IL-6 has been shown to be indispensable for pathogenic Th17 cell expansion leading to IL-17A overproduction and bone loss in ligature-induced periodontitis. Moreover, IL-6 is a signature gene induced by IL-17A signaling in many cell types, thus establishing a feed-forward amplification cycle of local inflammation Consistent with this, we saw that the local delivery of anti-IL-17A MP downregulated 116 mRNA expression in periodontal tissues, which may be central to its efficacy, interfering with this inflammatory loop in the periodontal tissues.

In conclusion, the current work demonstrates that local and sustained release of IL-17A Abs can halt inflammatory bone loss in murine PD, which served as a model for chronic osteolytic diseases. Accordingly, this approach may represent a promising therapeutic strategy for conditions in which inflammatory bone loss is driven by IL-17A mediated inflammation.

### Example 2

The local induction of M2 macrophages by local delivery of IL-4 to restore periodontal immune homeostasis and prevent periodontal inflammatory bone loss in mice models was evaluated. The following results show that this strategy is effective in halting periodontitis progression by inhibiting inflammatory bone loss and promoting inflammation resolution.

### Methods and Results

### Fabrication, Characterization and Functional Assessment of IL-4 and CCL2 Sustained Release PLGA Microparticles (MPs)

Recombinant mouse IL-4 and CCL2 protein were encapsulated in poly-lactic-co-glycolic acid (PLGA) microparticles (MPs) using a double emulsion technique. The IL-4 PLGA MPs were surface characterized using scanning electron microscope (SEM), and their size distribution was determined using volume impedance measurements. Then there was a determination of the functional bioactivity of IL-4 protein released from the MPs compared to the soluble protein by assessing arginase activity in RAW 264.7 cells, and flow cytometric analysis of M2 macrophages markers in murine bone marrow derived macrophages (BMDMs). In addition, there was an assessment of the release profile of IL-4 from the PLGA MPs over a period of 30 days using ELISA.

SEM evaluation of IL-4 MPs showed uniformly spherical MPs with moderate surface porosity (FIG. 6A). The mean size of IL-4 MPs was 23.09±12.22 um as determined by volume impedance measurements. Furthermore, ELISA release profile data show that the IL-4 MPs continued to be sustainably released from MPs over a period of 30 days, with about 85% of the cytokine released over the first 5 days (FIG. 6B). In vitro functional assays showed that conditioned medium containing IL-4 released from MPs upregulated arginase activity (M2 marker) in RAW cells (FIG. 6C) and increased expression of CD206 (surface marker of M2 macrophages) in the same manner as soluble IL-4 (FIG. 6D).

### Assessment of the Therapeutic Effect of IL-4 MPs in Murine Periodontal Disease Models

To further validate the protective effect of IL-4 in murine periodontitis models and test whether this effect is dose dependent, there was performed a series of in vivo studies that tested the therapeutic effects of IL-4 MPs in three models of murine ligature periodontitis. The purpose of conducting the studies was to evaluate the therapeutic effects of IL-4 in conditions that mimic clinical scenarios, where most patients present with an already active periodontal disease and varying degree of tissue destruction. To that end, there was designed three subsets of in vivo experiments where IL-4 MPs were delivered either at the start (preventive model), midway (interventional model) or at the end of the disease induction phase (reparative model).

Using microCT analysis, it was shown that both CCL2 and IL-4 MPs inhibited bone loss significantly in both the preventive and interventional models (FIGS. 7A and 7B). Moreover, only IL-4 MPs accelerated bone gain in the reparative model (FIG. 8).

Evaluation of Macrophages and Osteoclasts Marker Expression In Vivo Using qPCR Analysis (FIG. 9)

Furthermore, there was performed qPCR analysis of RNA samples extracted from mice gingival tissues where IL-4 MPs were delivered, to analyze the relative expression of M1 versus M2 macrophages markers. IL-4 MPs upregulated the expression Arg-1 and IL-IRA expression (M2 macrophages markers), and downregulated iNOS expression (M1 macrophages marker) (FIG. 9).

In addition, both CCL2 and IL-4 MPs downregulated the expression of RANKL, a key ligand for osteoclastic cells differentiation, which may explain, at least in part, the inhibition of bone loss observed when CCL2 or IL-4 MPs were delivered and further confirms previous TRAP staining data indicating reduced numbers of TRAP+ cells (osteoclasts) in sections from CCL2 group. Since B and T cells are the primary source of RANKL under inflammatory conditions, the inhibitory effect of CCL2 on RANKL expression may imply that the in vivo biological effects of this chemokine extend beyond macrophages.

Finally, CCL2 delivery did not have any effect on FOXP3 expression, while IL-4 slightly upregulated FOXP3 gene expression. FOXP3 is a marker for CD4+ CD25+ regulatory T-cells (Tregs) known for their ability to induce tolerance and modulate inflammatory response. Conversely, along the same lines, neither CCL2 nor IL-4 had any effect on the expression of CCL22, a marker for M2A macrophages that is known to recruit T-regulatory cells locally.

### Conclusion

The in vitro and in vivo data demonstrate the feasibility of local induction of M2 macrophages as a promising strategy for restoring periodontal immune homeostasis and protecting against inflammatory bone loss.

### Example 3

A—In vivo Assessment of M2 Macrophages Local Induction and Inflammatory and Osteoclastic Markers Expression in Response to CCL2 and IL-4 MPs Delivery in Murine Gingival Tissues

A1—Fluorescent Activated Cell Sorting (FACS) for Murine Gingival Macrophages

To evaluate the shifts in macrophages phenotype in response to local delivery of CCL2 or IL-4 MPs, ligature induced periodontitis was induced for 7 days in 8-10 male Balb/C mice. The mice were anesthetized by intraperitoneal injection of 80 mg/8 mg/kg Ketamine/Xylazine, then, under a stereomicroscope, 6-0 silk suture were ligated around the left maxillary second molar with the knot placed on the palatal side. Following ligatures placement, 10 mg PLGA MPs were suspended in 1 ml of 2% carboxymethylcellulose (CMC) in PBS. Then, 50 ul of the MPs suspension were locally injected in the gingival tissues around the ligated tooth using 29½ G insulin needles as follows: 20 ul on the buccal gingiva, and 30 ul dispersed equally on the mesial, middle and distal aspects of the ligated maxillary second molar palatal gingiva. At the same day of ligatures placement, either CCL2 or IL-4 MPs were locally delivered on the buccal and palatal aspects of the ligated tooth. Mice with ligatures only, ligatures with Blank MPs injection or age matching healthy mice were used as controls.

After 7 days from ligatures placement with or without MPs injection, mice in all groups were sacrificed and mice maxillae were harvested for gingival cells isolation as described previously. Alveolar gingival tissues surrounding the ligated tooth were dissected and minced then digested in 5 ml RPMI with 3.2 mg/ml Collagenase Type IV and 1 mg/ml DNAse for 1 hour. In the last five minutes of digestion, 50 ul of EDTA was added to the digest. Next, 5 ml of RPMI with 1 mg/ml DNAse was added to the collagenase DNAse medium containing digested gingival tissue, then the mixture was be passed through 70 μm cell strainer to obtain a single cell suspension. The cell suspension was then centrifuged at 1500×g for 6 minutes in a pre-cooled centrifuge, then the cell pellet was re-suspended in PBS for cell surface and intracellular staining.

To identify phenotypic changes of macrophages in gingival tissues, a gating strategy was used. It started by positive gating on live cells as indicated by a viability marker, Sfeir, Charles R21DE025735 Treatment of periodontitis by homing M2 macrophages followed by negative gating on neutrophils (LY6G+), T cells (TCRβ+) and B cells (B220+). Next, there was positive gated on cells expressing the hematopoietic marker CD45 (common leukocyte marker). From the CD45+ population, there was identified macrophages subsets by positive gating on M1 macrophages (CD45+, CD11b+ and CD86+) and M2 macrophages (CD45+, CD11b+ and CD206+).

### Results

A pilot study (n=3) was conducted to compare the shifts in macrophages phenotype between mice with only ligature induced periodontitis and healthy mice. The data showed that PD induction with ligature for 7 days results in skewing of gingival macrophages towards an M1 like phenotype (CD45+, CD11b+ and CD86+) (FIG. 10A). Furthermore, PD induction causes a slight reduction in the number of M2 like macrophages (CD45+, CD11b+ and CD206+) in the gingiva (FIG. 10B).

A-2 Quantitative Real Time Polymerase Chain Reaction (qPCR) Analysis for Inflammatory and Osteoclastic and Macrophages Polarization Markers

For qPCR analysis, the 7 days ligature PD experiment described above was repeated and samples were collected for RNA extraction and qPCR analysis. Mice were sacrificed at day 4 and day 7 post-ligature placement with or without local MPs injection. Following mice sacrifice, the half-maxillae on the ligated side was harvested and flash frozen in liquid nitrogen then stored in −80 □C until the day of RNA extraction. For RNA extraction, the frozen half maxillae samples with be first thawed at 4° C. in RNA later solution overnight. The next day, the alveolar gingiva on the ligated side with be dissected under magnification from each half maxilla. The dissected gingiva was then homogenized in Trizol reagent (Thermofisher), then vortexed with chloroform and centrifuged at 12000×g at 4° C. for 15 minutes. Following centrifugation, the clear top layer (containing RNA) with be transferred to RNeasy minicolumns for RNA purification using RNeasy Mini kit (Qiagen). The RNA concentration was measured with Nanodrop then reverse transcribed to cDNA using High-Capacity RNA-to-cDNA™ Kit (Applied biosystems). Quantitative polymerase chain reaction was conducted with TaqMan™ Gene Expression Master Mix (Applied Biosystems) on a QuantStudio 6 Flex Real-Time PCR system (Thermo Fisher Scientific), and all primers and probes were purchased from TaqMan (Applied Biosystems). Data analysis was done with the delta-delta Ct method. The expression of the following genes will be assessed: Arg1 and IL-1ra (M2 macrophages), NOS2 and IL6 (M1 macrophages), IL17a (Th17), FOXP3 (Tregs), ACP5 (TRAP—Osteoclasts) and TNFSF11 (RANKL—Osteoblasts derived osteoclasts differentiation factor).

### Results

The in vivo experiment described above was conducted with mice maxillae harvested for RNA extraction at 4 and 8 days. Following RNA extraction and reverse transcription to cDNA, we performed qPCR analysis for Arg-1 (M2 marker), NOS2, IL-6 (M1 markers) and RANKL (osteoclasts differentiation factor).

At 4 days (FIG. 11A), only mice with IL-4 MPs delivery exhibited upregulated Arg-1 expression compared to the untreated control. On the other hand, mice in the IL-4 and CCL2 MPs showed downregulated expression of NOS2 and IL-6 compared to untreated group. Finally, only CCL2 MPs downregulated the expression of RANKL compared to the untreated group.

At 8 days (FIG. 11B), IL-4 MPs enhanced the expression of Arg-1, while both IL-4 and CCL2 MPs inhibited the expression of RANKL compared to untreated mice. No significant difference in NOS2 expression was observed between all experimental groups at 8 days.

### A3—Evaluation of M2 Macrophages Local Induction as a Preventive Therapeutic Approach for Murine Periodontitis

Analysis of inflammatory bone loss in murine ligature periodontitis following a preventive M2 induction therapy: the 7 days ligature PD murine model described above was repeated with rm CCL2 or IL-4 MPs delivered on the same day of ligature placement (Preventive therapeutic approach). Similar to the sorting and qPCR experiments, age matching healthy mice, mice with ligature only or with ligatures and Blank MPs delivery will be used as controls. Table 1 describes the experimental groups used in the preventive approach experiment.

TABLE 1
Description of the experimental groups used in the preventive therapeutic
approach experiment.

| Groups | Healthy | | | | |
|---|---|---|---|---|---|
| (n = 6 mice) | Control | Untreated | Blank MPs | IL-4 MPs | CCL2 MPs |
| Description | No ligature | 2^{nd} molar | 2^{nd} molar Ligature for 7 | 2^{nd} molar Ligature for | 2^{nd} molar |
| | and no MPs | Ligature for 7 | days + Blank MPs | 7 days + IL-4 MPs | Ligature for 7 |
| | delivery | days | delivery at day 0 | delivery at day 0 | days + CCL2 MPs |
| | | | | | delivery at day 0 |

After 7 days from ligatures placement with or without MPs delivery, were be sacrificed, and maxillae were be harvested fixed overnight in 10% neutral buffered formalin then transferred to 70% ethanol for scanning with a micro-computed tomography system. (Scanco μCT 50, Scanco Medical). A resolution of 10μm voxel size, 55 KVp, 0.36 degrees rotation step (180 degrees angular range) and a 1200 ms exposure per view will be used for the scans. All scans were reoriented with DataViewer software (GE Healthcare) to a standardized orientation guided by pre-defined anatomical landmarks. For bone loss evaluation, reoriented images were used to measure the distance between the cementoenamel junction (CEJ) of the maxillary second molar and the level of the alveolar bone crest (ABC) on the mesial, distal, buccal and palatal sides using CTAN software (Bruker). For mesial and distal measurements, the CEJ to ABC distance was measured on 8 measurement slices with a distance interval of 40 μm in between. For the buccal and palatal measurements, the CEJ to ABC distance was measured on 20 measurement slices with a distance interval of 30 μm in between.

### Results

The results (FIGS. 12A and 12B) showed that mice with either IL-4 or CCL2 MPs delivery had significant inhibition of alveolar bone loss around the ligated second molar compared to mice with ligature only (FIG. 7D) or mice with Blank MPs injection. This inhibition was most prominent on the proximal (average of mesial and distal) aspect of the ligated second molar. The ligated molar buccal and palatal aspects in all groups showed no statistical difference in all groups. The total bone loss inhibition on all aspects was statistically significant in both IL-4 and CCL2 MPs groups when compared to the ligature only or Blank MPs groups.

### B. Assessing the Protective and Pro-Reparative Effects of M2 Macrophages Induction in Murine Ligature Periodontitis Progression and Resolution

The data from FIGS. 12A and 12B shows the therapeutic effect of CCL2 when injected at the start of the ligature placement, at the initiation of the disease. At that stage, CCL2 or IL-4 sustained release MPs were planned to be delivered at the same time of ligature placement while the inflammatory response and bone loss have not been established yet (Preventive approach). In this aim, the M2 induction therapy was started either at the progression stage (Interventional approach) or at the recovery stage (Reparative approach) of murine ligature periodontitis. For the interventional approach, CCL2 or IL-4 MPs were delivered after 4 days from ligature placement in a 10 days ligature periodontitis model. For the reparative approach, CCL2 or IL-4 MPs were delivered on the same day of ligature removal after 10 days of ligature induced periodontitis. The interventional and reparative approaches experiments tested the therapeutic strategy in the presence of an active inflammation or advanced tissue destruction. Both testing conditions mimic real-life clinical scenarios.

### B1—Evaluation of M2 Macrophages Local Induction as an Interventional Therapeutic Approach for Murine Periodontitis

Ligature periodontitis was induced in 8-10 weeks old male Balb/C mice as described previously. The disease induction period lasted for 10 days with CCL2, IL-4 or blank MPs locally delivered in the gingiva surrounding the ligated tooth after 4 days from ligature placement. As controls, Age matching healthy control mice and mice with ligature periodontitis only for 4 and 10 days were used as described in the Table 2 below.

TABLE 2
Description of the experimental groups for the interventional approach
experiment.

| Group | Healthy | | Lig. 10D | Lig. 4D + | Lig. 4D + IL-4 | Lig. 4D + |
|---|---|---|---|---|---|---|
| (n = 6 mice) | Control | Lig. 4D | (Untreated) | Blank MPs | MPs | CCL2 MPs |
| Description | No ligature | 2nd molar | 2nd molar | 2nd molar | 2nd molar | 2nd molar |
| | and no MPs | Ligature for 4 | Ligature for 10 | Ligature for | Ligature for | Ligature for |
| | delivery | days | days | 10 days + | 10 days + IL- | 10 days + |
| | | | | Blank MPs at | 4 MPs at day | CCL2 MPs at |
| | | | | day 4 | 4 | day 4 |

At the end of disease induction period, mice will be sacrificed, and maxillae were harvested and fixed overnight in 10% neutral buffered formalin then transferred to 70% ethanol for scanning with a micro-computed tomography system. Analysis of alveolar bone was performed by measuring the distance between the cementoenamel junction (CEJ) of the maxillary second molar and the level of the alveolar bone crest (ABC) on the mesial, distal, buccal and palatal sides using the same protocol described in section A3.

### Results

FIGS. 13A, 13B and 13C show that mice with either IL-4 or CCL2 MPs delivery had significant inhibition of proximal bone loss when compared to mice with ligature only (FIG. 10D) or mice with Blank MPs injection. On the buccal aspect, bone loss inhibition by CCL2 or IL-4 MPs was only statistically different from the ligature only, but not the Blank group. On the palatal aspect, only IL-4 produced statistically significant inhibition of bone loss compared to the ligature only and the Blank groups. The total bone was statistically different between both IL-4 and CCL2 MPs groups and the untreated group. However, only IL-4 MPs group showed statistically significant inhibition compared to the Blank group for total bone loss.

### B2: Evaluation of M2 Macrophages Local Induction as a Reparative Therapeutic Approach for Murine Periodontitis

### Analysis of Inflammatory Bone Loss Following Reparative M2 Induction Therapy for Murine Ligature Periodontitis:

Ligature periodontitis was induced for 10 days in 8-10 weeks old male Balb/C mice as described previously. At the end of the 10 days disease induction period, ligature were removed concurrently with local delivery of Blank, CCL2 or IL-4 MPs in the gingiva surrounding the previously ligated tooth. Mice with ligature removal without

MPs delivery and age matching healthy control mice were used as controls. For the purpose of data normalization, there was added an additional control group of mice with only the induction of ligature periodontitis for 10 days. Table 3 summarizes the experimental groups used in the experiment.

TABLE 3
Description of the experimental groups for the reparative approach experiment.

| | | | Lig. 10D + 4D | Blank MPs | IL-4 MPs | CCL2 MPs |
|---|---|---|---|---|---|---|
| | Healthy | | Recov. | (D10) | (D10) | (D10) |
| Group | Control | Lig. 10D | (Untreated) | Lig. 10 D + 4D Recov. | | |
| Description | No ligature | Ligature PD | Ligature PD for | Ligature PD | Ligature PD | Ligature PD |
| | and no MPs | for 10 days | 10 days + 4 days | for 10 days + | for 10 days + | for 10 days + |
| | delivery | | recovery post | Blank MPs at | IL-4 MPs at | CCL2 MPs at |
| | | | ligature removal | day 10 + 4 | day 10 + 4 | day 10 + 4 |
| | | | | days | days | days |
| | | | | recovery post | recovery post | recovery post |
| | | | | ligature | ligature | ligature |
| | | | | removal | removal | removal |

At the end of the 4 days recovery period, mice were sacrificed, and maxillae were harvested and processed for micro-computed tomography scanning. Analysis of alveolar bone loss was done by measuring the distance between the cementoenamel junction (CEJ) of the maxillary second molar and the level of the alveolar bone crest (ABC) on the mesial, distal, buccal and palatal sides using the same protocol described in A3.

To illustrate the amount of alveolar bone gained during the 4 days recovery period after ligature removal, measurements on each aspect from all groups was normalized to the average measurements of the 10 days ligature only group which was used as baseline.

FIGS. 14A, 14B and 14C indicate that both CCL2 and IL-4 MPs significantly accelerate proximal alveolar bone gain following ligature removal. The difference in bone gain on the buccal and palatal aspects of the 2n^{d} molar was not statistically significant between all groups that had undergone the 4 days recovery period. The total alveolar bone gain on all aspects was significantly higher for IL-4 MPs group when compared with the ligature removal only or the Blank MPs groups. The total bone gain in the CCL2 MPs group was significantly higher than the Blank MPs group only.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A microparticle, comprising:
degradable polymer; and
a compound selected from the group consisting of C-C motif chemokine ligand 2 (CCL2), interleukin 4 (IL-4), anti-interleukin 17 (IL17 antibody), and mixtures or blends thereof,
wherein the degradable polymer is loaded with the compound, and the microparticle is structured to provide a sustained, controlled release of said compound from said degradable polymer.

2. The microparticle of claim 1, wherein the degradable polymer comprises poly(lactic-co-glycolic) acid.

3. The microparticle of claim 1, wherein said microparticle is administered in a therapeutically effective amount to a patient.

4. The microparticle of claim 1, wherein said microparticle is an active ingredient in a composition.

5. The microparticle of claim 4, wherein the microparticle is administered by locally injecting the composition in the periodontium of a patient.

6. The microparticle of claim 1, wherein said microparticle is effective to reduce or eliminate periodontal disease and reduce inflammation.

7. The microparticle of claim 1, wherein the compound is anti-interleukin 17 (IL-17 antibody) and said microparticle is effective to prevent osteoclast formation for the prevention of bone loss.

8. The microparticle of claim 1, wherein the compound is at least one of C-C motif chemokine ligand 2 (CCL2) and interleukin 4 (IL-4), and said microparticle is effective to recruit M2 macrophages and convert M1 macrophages to M2 for prevention of inflammation.

9. A pharmaceutical composition, comprising:
loaded microparticles, comprising:
degradable polymer; and
a compound selected from the group consisting of C-C motif chemokine ligand 2 (CCL2), interleukin 4 (IL-4), anti-interleukin 17 (IL17) antibody, and mixtures or blends thereof; and
at least one ingredient selected from the group consisting of pharmaceutically acceptable carrier, adjuvant and excipient.

10. A method, comprising:
administering a therapeutically effective amount of a compound selected from the group consisting of C-C motif chemokine ligand 2 (CCL2), interleukin 4 (IL-4), anti-interleukin 17 (IL17 antibody), and mixtures or blends thereof, to a subject in need of reduced tissue inflammation, wherein the compound is locally delivered at the site of the tissue inflammation in a sustained release form, and the sustained release form comprises microparticles loaded with the compound.

11. The method of claim 10, wherein the tissue inflammation is periodontal disease.

12. A method of treating a patient with tissue inflammation, comprising:
forming microparticles, comprising:
degradable polymer; and
a compound selected from the group consisting of C-C motif chemokine ligand 2 (CCL2), interleukin 4 (IL-4), anti-interleukin 17 (IL17 antibody), and mixtures or blends thereof,
wherein the degradable polymer is loaded with the compound; and
administering a therapeutically effective amount of the microparticles locally to the tissue inflammation,
wherein the microparticles are structured to provide a sustained, controlled release of said compound from said degradable polymer to the tissue inflammation.

13. The method of claim 12, wherein the method is effective in clinical scenarios, comprising:
preventative, wherein tissue is healthy, and disease is induced but microparticles are also delivered, the disease is prevented from occurring;
interventional, wherein tissue is inflamed, disease is in progress, then the microparticles are injected, and the disease is halted or stopped; and
wherein the cause of the disease is removed, removal of the ligature, then microparticles are injected, and healing and regeneration at the site is enhanced.
